(19) United States
(12) Patent Application Publication
(10) Pub. No.: US 2024/0026418 A1
(43) Pub. Date: Jan. 25, 2024

(54) BACTERIAL GENE-ASSOCIATED METHODS AND COMPOSITIONS FOR DIAGNOSING AND TREATING COLORECTAL CANCER

(71) Applicant: FRED HUTCHINSON CANCER CENTER, Seattle, WA (US)

(72) Inventors: Neelendu Dey, Seattle, WA (US); Samuel Minot, Seattle, WA (US)

(21) Appl. No.: 18/320,878

(22) Filed: May 19, 2023

Related U.S. Application Data
(60) Provisional application No. 63/344,523, filed on May 20, 2022.

(51) Int. Cl. C12Q 1/10; G16B 30/10; A61K 45/06; C12Q 1/6886; G16H 50/30

(52) U.S. Cl. CPC C12Q 1/10; G16B 30/10; A61K 45/06; C12Q 1/6886; G16H 50/30; C12Q 2600/118

(57) ABSTRACT

The present disclosure provides compositions and non-invasive methods for diagnosing and treating a subject at risk for developing, or having, or at risk for progressing on colorectal cancer (CRC) based on analysis of bacterial species in the gut microbiome of the subject.

### STATEMENT OF GOVERNMENT INTEREST

This invention was made with government support under DK111941 awarded by the National Institutes of Health. The government has certain rights in the invention.

### BACKGROUND

Colorectal cancer (CRC) is one of the most common cancers globally. The majority of CRC cases presently cannot be linked to hereditary or familial drivers. Current first-tier screening strategies, colonoscopy and fecal immunochemical test (FIT), are effective, but imperfect. Colonoscopy, the bedrock of the US CRC screening strategy, has established the value of targeting early precursor lesions of CRC: colorectal adenomas (polyps). However, cost, access, socio-economic marginalization, cultural and/or language factors, and rural residence are all barriers to colonoscopy uptake. Disproportionate CRC burden is suffered by minority communities including Black Americans and Alaska Natives. Non-invasive testing may lower costs and have greater uptake. Indeed, systematic deployment of FIT has been demonstrated to increase screening rates and decrease CRC-related mortality. However, meta-analyses have found that FIT sensitivity for detecting CRC is moderate (pooled sensitivity 79%), and sensitivity for detecting advanced adenomas (adenomas designated as high-risk based on size and/or histology) is low (pooled sensitivity 40%). There is a public health need for novel high-sensitivity clinical tools for early detection of CRC and precursor lesions.

The gut microbiome is an emerging environmental risk factor for CRC (see Burkitt, Cancer 28 1971, Klein et al, NEJM 1977, Toprak et al, Clinical Microbiology and Infection 2006, Wang et al, Cancer Research 2008, Swidsinski et al, Gastroenterology 1998, Kostic et al, Genome Research 2012, Long et al, Nature Microbiology 2019, and Wirbel et al, Nature Medicine 2019). While several specific gut microbes have been identified as potentially carcinogenic, each appears to be causative in a small minority of CRC cases, and in those cases, estimated effect sizes are modest.

### BRIEF DESCRIPTION OF THE FIGURES

FIG. 1 shows a heat map of identified genes grouped by association with CRC for various strains of Blautia obeum.

FIGS. 2A and 2B relate to meta-analysis of gut microbiome surveys from global CRC cohorts (pooling published metagenomic datasets).

FIG. 3 shows (left) Wald test association with CRC versus proportional abundance of selected genes, and (right) an example calculation of CRC-association scores.

FIG. 4 show taxonomic classification of each CAG estimated by aligning against the NCBI RefSeq genome collection.

FIG. 5 shows a graph of bacterial genomes of gut bacteria that exhibit a CRC Wald statistic >1 (top) and a CRC Wald statistic <−1 (bottom).

FIGS. 6A and 6B show cancer-associated and health-associated bacteria based on Wald statistical analysis for use in designing bacterial consortia.

FIGS. 7A-7C show results of the CRC-associated bacterial consortia in a preclinical mouse model. In FIG. 7C, for each condition (Gdf15, Cdkn2α, Ifng), the left of the two bars is “anti-tumor” and the right of the two bars is “pro-tumor”.

FIG. 8 shows multiple cell types expressing senescence genes as determined from single cell RNA sequencing.

### DETAILED DESCRIPTION

The present disclosure generally relates to diagnosing CRC, risk-profiling CRC, and treating a subject with CRC based on analysis of bacterial species and/or the presence and/or prevalence and/or amount of bacteria comprising certain genes in the gut microbiome of the subject.

The present disclosure provides, for the first time, that analyzing gene content of gut bacteria in terms can reveal CRC risks. Microbiomes with cancer-associated gene signatures induce greater tumor burden in a mouse model of CRC. Without being bound by theory, the microbiome may influence CRC risk via field effects.

Presently disclosed associations, methods, and compositions support microbiome-based risk profiling and non-invasive screening for CRC. Faecal Immunochemical Tests (FITs) comprise sufficient residual stool to profile and score the microbiome in accordance with the present disclosure. Profiling the microbiome may predict, identify, and/or interrogate precancerous changes (e.g. field effects). Non-invasive microbiome-based testing may help meet the unmet need of improving population-wide screening.

The present disclosure includes the following, non-limiting, enumerated Embodiments.

Embodiment 1. A method for identifying a subject as being at-risk for developing, as having, or as being at-risk for progressing on colorectal cancer (CRC), the method comprising detecting, in a fecal sample from the subject, the presence of one or more organism from Table 1 (e.g., from Column A of Table 1) having a Mean CRC Wald score greater than zero in Column B of Table 1, wherein the subject is identified as at-risk for developing CRC or as having CRC or as at-risk for progressing on CRC when the one or more organism is present in the fecal sample.

Embodiment 2. A method for identifying a subject as being at-risk for developing, as having, or as being at-risk for progressing on colorectal cancer (CRC), the method comprising, the method comprising: (a) determining whether one or more organism from Table 1 (e.g., from Column A of Table 1) having a Mean CRC Wald score greater than zero in Column B of Table 1 is more abundant in the fecal sample than one or more organism from Table 1 (e.g., from Column A of Table 1) having a Mean CRC Wald score less than zero in Column B of Table 1; and (b) determining that the subject is at-risk for developing or has or is at-risk for progressing on CRC when one or more organism from Table 1 (e.g., from Column A of Table 1) having a Mean CRC Wald score greater than zero in Column B of Table 1 is more abundant in the fecal sample than one or more organism from Table 1 (e.g., from Column A of Table 1) having a Mean CRC Wald score less than zero in Column B of Table 1.

Embodiment 3. A method for identifying a subject as being at-risk for developing, as having, or as being at-risk for progressing on colorectal cancer (CRC), the method comprising: (a) detecting a fecal metagenome in a fecal sample from the subject; and (b) comparing (i) the amount or prevalence, in the fecal sample, of one or more organism from Table 1 (e.g., from Column A of Table 1) having a Mean CRC Wald score greater than zero in Column B of Table 1 (e.g., from Column A of Table 1) with (ii) the amount or prevalence of the one or more organism in a reference fecal sample from a non-CRC subject, and/or with (iii) the mean or median amount or prevalence of the one or more organism across a plurality of reference fecal sample from non-CRC subjects, wherein an increase in (i) as compared to (ii) and/or to (iii) identifies the subject as being at-risk for developing for or progressing on CRC, or as having CRC.

In some embodiments, a reference subject is of the same gender, ethnicity, overall health, and/or age of the subject (e.g., ±1, 2, 3, 4, 5, 6, 7, 8, 9, or 10 years of the age of the subject). A prevalence or amount of an organism can be determined, for example, using one or more labelled or otherwise detectable antibodies specific for an organism of interest or specific for a target (e.g. protein, carbohydrate, glycoprotein, lipid, glycolipid) produced by or associated with the organism of interest, or using nucleic acid amplification reagents and an amplification process (e.g., qPCR) specific for the organism (e.g., amplifying one or more genomic markers specific to or otherwise identifying the organism).

Embodiment 4. A method for selecting a compound or composition (e.g., for use in treating or preventing or delaying onset of colorectal cancer (CRC) in a subject), the method comprising: contacting a candidate compound or composition, or a plurality of candidate compounds or compositions (e.g., from a library), with: (i) one or more organism from Table 1 (e.g., from Column A of Table 1) having a Mean CRC Wald score greater than zero in Column B of Table 1; and/or (ii) one or more organism from Table 1 (e.g., from Column A of Table 1) having a Mean CRC Wald score less than zero in Column B of Table 1, for a time and under conditions sufficient to determine whether the compound inhibits growth and/or activity of, or kills the one or more organism, of (i) and/or whether the compound promotes growth and/or activity of the one or more organism of (ii), and selecting a compound or composition that inhibits growth and/or activity of, or kills the one or more organism, of (i) and/or that promotes growth and/or activity of the one or more organism of (ii).

Promoting growth and/or activity, and inhibiting growth and/or activity, or killing, can be assessed by a growth, activity, or killing assay known to those of ordinary skill in the art. For example, a viability or growth assay (e.g., under culture conditions appropriate to the one or more organism) may be used. Activity may be assessed by, for example, assaying for the presence or absence of motility (if the one or more organism is typically motile) and/or the presence or absence of a product known to be typically produced by the one or more organism.

Embodiment 5. A method for treating or managing colorectal cancer (CRC), the method comprising, to a subject identified as being at-risk for developing or for progressing on colorectal cancer (CRC) by the method of any one of Embodiments 1-3: (i) prescribing and/or performing a colonoscopy; and/or (ii) prescribing and/or performing increasing a number and/or a frequency of colonoscopies; and/or (iii) prescribing and/or performing a colon resection surgery; and/or (iv) removing one or more polyp; and/or (v) prescribing a NSAID, such as aspirin; and/or (vi) prescribing a plant-based diet or prescribing an increase in the plant content of the subject's diet; and/or (vii) prescribing and/or administering a compound identified by the method of Embodiment 4; and/or (viii) manipulating the gut microbiome of the subject, such as, for example, by administering one or more probiotic and/or performing a fecal transplant such that, in a subsequent fecal sample from the subject, the prevalence of one or more organism from Table 1 (e.g., from Column A of Table 1) having a Mean CRC Wald score greater than zero in Column B of Table 1 is decreased relative to the prevalence of one or more organism from Table 1 (e.g., from Column A of Table 1) having a Mean CRC Wald score less than zero in Column B of Table 1, relative to the respective prevalences prior to the manipulation.

In some embodiments, a subject identified as being at-risk for developing or for progressing on colorectal cancer (CRC) according to a disclosed method receives chemotherapy, immunotherapy (e.g., comprising a therapeutic antibody and/or a therapeutic immune cell), radiation therapy, proton therapy, colon resection surgery, or any combination thereof).

Embodiment 6. A method for monitoring colorectal cancer (CRC) in a subject, the method comprising determining whether a fecal sample of the subject comprises (i) a greater or a lesser amount or prevalence of one or more organism from Table 1 (e.g., from Column A of Table 1) having a Mean CRC Wald score greater than zero in Column B of Table 1, as compared to a previous fecal sample from the subject, and/or (ii) an increased or a decreased ratio of [one or more organism from Table 1 (e.g., from Column A of Table 1) having a Mean CRC Wald score greater than zero in Column B of Table 1] to [one or more organism from Table 1 (e.g., from Column A of Table 1) having a Mean CRC Wald score less than zero in Column B of Table 1], as compared to a previous fecal sample from the subject.

Embodiment 7. The method of any one of Embodiments 1-6, further comprising obtaining the fecal sample from the subject.

Embodiment 8. A kit for identifying a subject as being at-risk for developing, as having, or as being at-risk for progressing on colorectal cancer (CRC), the kit comprising: (1) a reagent for typing or for identifying one or more organism from Table 1 (e.g., from Column A of Table 1) having a Mean CRC Wald score greater than zero in Column B of Table 1, and, optionally, (2) a reagent for typing or for identifying one or more organism from Table 1 (e.g., from Column A of Table 1) having a mean CRC Wald score less than zero in Column B of Table 1, wherein the reagent of (1) and/or (2) is optionally selected from the group consisting of: (i) one or more nucleic acid probe capable of hybridizing with a genomic nucleic acid sequence from one or more organism from Table 1 (e.g., from Column A of Table 1), wherein, preferably, the genomic nucleic acid sequence is present in a Genome Assembly Accession according to Column C of Table 1; (ii) a forward and a reverse nucleic acid primer capable of amplifying a genomic nucleic acid from one or more organism from Table 1 (e.g., from Column A of Table 1), wherein, preferably, the genomic nucleic acid sequence is present in a Genome Assembly Accession according to Column C of Table 1, and (iii) one or more antibody specific for the one or more organism from Column B (e.g., one or more organism from Column A of Table 1 identified by a Mean CRC Wald score as in Column B of Table 1) of Table 1; and instructions for using the reagent(s) to identify the presence or an increased presence of one or more organism from Table 1 (e.g., from Column A of Table 1) having a Mean CRC Wald score greater than zero in Column B of Table 1.

Embodiment 9. The method of any one of Embodiments 1-7 and 12 or the kit of Embodiment 8, wherein the one or more organism from Table 1 (e.g., from Column A of Table 1) having a Mean CRC Wald score greater than zero in Column B of Table 1 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more organisms from Table 1 (e.g., from Column A of Table 1) having a Mean CRC Wald score greater than zero in Column B of Table 1.

Embodiment 10. The method of any one of Embodiments 2, 4, 5, 6, 7, 9, and 12 or the kit of Embodiment 8, wherein the one or more organism from Table 1 (e.g., from Column A of Table 1) having a Mean CRC Wald score less than zero in Column B of Table 1 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more organisms from Table 1 (e.g., from Column A of Table 1) having a Mean CRC Wald score less than zero in Column B of Table 1.

Embodiment 11. The method of any one of Embodiments 1-7 and 9-10 or the kit of any one of Embodiments 8-10, wherein the one or more organism from Table 1 (e.g., from Column A of Table 1) having a Mean CRC Wald score greater than zero in Column B of Table 1 has a Mean CRC Wald score greater than 0.01, greater than 0.05, greater than 0.1, greater than 0.5, greater than 1, or greater than 2.

Embodiment 12. A method of treating colorectal cancer (CRC) in a subject, the method comprising administering to the subject an effective amount of:
(1) a compound or composition that inhibits growth and/or activity of, or kills one or more organism from Table 1 (e.g., from Column A of Table 1) having a Mean CRC Wald score greater than zero in Column B of Table 1; and/or
(2) a compound or composition that promotes growth and/or activity of one or more organism from Table 1 (e.g., from Column A of Table 1) having a Mean CRC Wald score less than zero in Column B of Table 1.

In some embodiments, the compound or composition of (1) specifically or preferentially inhibits or kills the one or more organism from Table 1 (e.g., from Column A of Table 1) having a Mean CRC Wald score greater than zero in Column B of Table 1, and does not inhibit or kill, or does not substantially inhibit or kill, one or more other organism present in a fecal sample of the subject (e.g., one or more organism from Table 1 (e.g., from Column A of Table 1) having a Mean CRC Wald score less than zero in Column B of Table 1). In some embodiments, the compound or composition of (2) specifically or preferentially promotes growth and/or activity of one or more organism from Table 1 (e.g., from Column A of Table 1) having a Mean CRC Wald score less than zero in Column B of Table 1, and does not promote growth and/or activity, or does not substantially promote growth and/or activity, of one or more other organism present in a fecal sample of the subject (e.g., one or more organism from Table 1 (e.g., from Column A of Table 1) having a Mean CRC Wald score less than zero in Column B of Table 1). In other words, in certain embodiments, administering a compound or composition provides a relative effect of decreasing an amount and/or activity one or more CRC-associated organism as compared to the amount and/or activity of one or more health-associated organism.

Embodiment 13. A non-transitory computer readable medium comprising computer executable instructions that when executed cause a processor to: (1) determine and/or quantify the presence, amount and/or prevalence, in a fecal sample from a subject, of one or more organism from Table 1 (e.g., from Column A of Table 1) having a Mean CRC Wald score greater than zero in Column B of Table 1 (e.g., from Column A of Table 1); and/or (2) determine and/or quantify the presence, amount and/or prevalence, in a fecal sample from the subject, of one or more organism from Table 1 (e.g., from Column A of Table 1) having a Mean CRC Wald score less than zero in Column B of Table 1 (e.g., from Column A of Table 1), wherein, optionally, the fecal sample of (1) and the fecal sample of (2) are the same sample or were collected from the subject at the same time or were collected from the subject within a 24 hour period.

Embodiment 14. The non-transitory computer readable medium of Embodiment 13, further comprising computer executable instructions that when executed cause a processor (optionally, the processor of Embodiment 13) to generate a ratio of (i) the amount and/or prevalence of the one or more organism from Table 1 having a Mean CRC Wald score greater than zero in Column B of Table 1 to (ii) the amount and/or prevalence of the one or more organism from Table 1 having a Mean CRC Wald score less than zero in Column B of Table 1, in the fecal sample.

Embodiment 15. The non-transitory computer readable medium of Embodiment 13 or 14, further comprising computer executable instructions that when executed cause a processor (optionally, the processor of Embodiment 13 or 14) to pass an alert to a user that the subject is at-risk for CRC or for progressing on CRC when (a) the presence, amount and/or prevalence, in the fecal sample from a subject, of the one or more organism from Table 1 having a Mean CRC Wald score greater than zero in Column B of Table 1, is greater than: (b) the amount or prevalence of the one or more organism in a reference fecal sample from a non-CRC subject; and/or is greater than (c) the mean or median amount or prevalence of the one or more organism across a plurality of reference fecal sample from non-CRC subjects.

Embodiment 16. The non-transitory computer readable medium of Embodiment 14 or Embodiment 15, further comprising computer executable instructions that when executed cause a processor (optionally, the processor of Embodiment 13, 14, or 15) to pass an alert to a user that the subject is at-risk for CRC or for progressing on CRC when the ratio of (i) to (ii) in the fecal sample is greater than: (A) the ratio of (i) to (ii) in a reference fecal sample from a non-CRC subject; and/or (B) (iii) the mean or median ratio of (i) to (ii) across a plurality of reference fecal samples from non-CRC subjects.

Embodiment 17. The non-transitory computer readable medium of any one of Embodiments 14-16, further comprising computer executable instructions that when executed cause a processor (optionally, the processor of any one of Embodiments 14-16) to pass an alert to a user that the subject is not at-risk or for CRC or for progressing on CRC when the ratio of (i) to (ii) in the fecal sample is less than: (A) the ratio of (i) to (ii) in a reference fecal sample from a non-CRC subject; and/or (B) (iii) the mean or median ratio of (i) to (ii) across a plurality of reference fecal samples from non-CRC subjects.

Embodiment 18. The non-transitory computer readable medium of any one of Embodiments 15-17, wherein the user is at least one of a patient and a physician.

Embodiment 19. The non-transitory computer readable medium of any of Embodiments 15-18, wherein the alert is provided in at least one of an aural form or a visual form.

Embodiment 20. The non-transitory computer readable medium of any of Embodiments 15-19, wherein the alert is indicative of at least one of: (i) prescribing and/or performing a colonoscopy; and/or (ii) prescribing and/or performing increasing a number and/or a frequency of colonoscopies; and/or (iii) prescribing and/or performing a colon resection surgery; and/or (iv) removing one or more polyp; and/or (v) prescribing a NSAID, such as aspirin; and/or (vi) prescribing a plant-based diet or prescribing an increase in the plant content of the subject's diet; and/or (vii) prescribing and/or administering a compound identified by the method of Embodiment 4; and/or (viii) manipulating the gut microbiome of the subject, such as, for example, by administering one or more probiotic and/or performing a fecal transplant such that, in a subsequent fecal sample from the subject, the prevalence of one or more organism from Table 1 (e.g., from Column A of Table 1) having a Mean CRC Wald score greater than zero in Column B of Table 1 is decreased relative to the prevalence of one or more organism from Table 1 (e.g., from Column A of Table 1) having a Mean CRC Wald score less than zero in Column B of Table 1, relative to the respective prevalences prior to the manipulation.

Embodiment 21. The non-transitory computer readable medium of any of Embodiments 13-20, wherein:
(i) the one or more organism from Table 1 (e.g., from Column A of Table 1) having a Mean CRC Wald score less than zero in Column B of Table 1 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more organisms from Table 1 (e.g., from Column A of Table 1) having a Mean CRC Wald score less than zero in Column B of Table 1; and/or
(ii) the one or more organism from Table 1 (e.g., from Column A of Table 1) having a Mean CRC Wald score greater than zero in Column B of Table 1 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more organisms from Table 1 (e.g., from Column A of Table 1) having a Mean CRC Wald score greater than zero in Column B of Table 1.

Embodiment 22. The non-transitory computer readable medium of any of Embodiments 13-21, further comprising computer executable instructions that when executed cause a processor (optionally, the processor of any one of Embodiments 13-21) to display a user interface on a display, the user interface having a plurality of fields operable to receive input from a user, the input indicative of whether the subject is at risk of CRC or is at risk of progressing on CRC.

In some embodiments, the one or more organism from Table 1 having a Mean CRC Wald score greater than zero in Column B of Table 1 has a Mean CRC Wald score in Column B of Table 1 greater than 0.01, greater than 0.02, greater than 0.03, greater than 0.04, greater than 0.05, greater than 0.06, greater than 0.07, greater 0.08, than greater than 0.09, greater than 0.1, greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, greater than 2.0, greater than 2.1, greater than 2.2, greater than 2.3, greater than 2.4, or greater than 2.5. In some embodiments, one or more organism from Table 1 having a Mean CRC Wald score less than zero in Column B of Table 1 has a Mean CRC Wald score in Column B of Table 1 less than −0.01, less than −0.02, less than −0.03, less than −0.04, less than −0.05, less than −0.06, less than −0.07, less than −0.08, less than −0.09, less than −0.1, less than −0.2, less than −0.3, less than −0.4, less than −0.5, less than −0.6, less than −0.7, less than −0.8, less than −0.9, less than −1.0, less than −1.1, less than −1.2, less than −1.3, less than −1.4, less than −1.5, less than −1.6, less than −1.7, less than −1.8, less than −1.9, less than −2.0, less than −2.1, less than −2.2, less than −2.3, less than −2.4, less than −2.5, less than −2.6, less than −2.7, or less than −2.8.

In some embodiments, (1) the one or more organism from Table 1 having a Mean CRC Wald score greater than zero in Column B of Table 1 has a Mean CRC Wald score in Column B of Table 1 greater than 0.01, greater than 0.02, greater than 0.03, greater than 0.04, greater than 0.05, greater than 0.06, greater than 0.07, greater than 0.08, greater than 0.09, greater than 0.1, greater than 0.2, greater than 0.3, greater than 0.4, greater than 0.5, greater than 0.6, greater than 0.7, greater than 0.8, greater than 0.9, greater than 1.0, greater than 1.1, greater than 1.2, greater than 1.3, greater than 1.4, greater than 1.5, greater than 1.6, greater than 1.7, greater than 1.8, greater than 1.9, greater than 2.0, greater than 2.1, greater than 2.2, greater than 2.3, greater than 2.4, or greater than 2.5, and (2) the one or more organism from Table 1 having a Mean CRC Wald score less than zero in Column B of Table 1 has a Mean CRC Wald score in Column B of Table 1 less than −0.01, less than −0.02, less than −0.03, less than −0.04, less than −0.05, less than −0.06, less than −0.07, less than −0.08, less than −0.09, less than −0.1, less than −0.2, less than −0.3, less than −0.4, less than −0.5, less than −0.6, less than −0.7, less than −0.8, less than −0.9, less than −1.0, less than −1.1, less than −1.2, less than −1.3, less than −1.4, less than −1.5, less than −1.6, less than −1.7, less than −1.8, less than −1.9, less than −2.0, less than −2.1, less than −2.2, less than −2.3, less than −2.4, less than −2.5, less than −2.6, less than −2.7, or less than −2.8.

In some embodiments, the one or more organism from Table 1 having a Mean CRC Wald score greater than zero in Column B of Table 1 has a Mean CRC Wald score in Column B of Table 1 of about 0.01, of about 0.02, of about 0.03, of about 0.04, of about 0.05, of about 0.06, of about 0.07, of about 0.08, of about 0.09, of about 0.1, of about 0.2, of about 0.3, of about 0.4, of about 0.5, of about 0.6, of about 0.7, of about 0.8, of about 0.9, of about 1.0, of about 1.1, of about 1.2, of about 1.3, of about 1.4, of about 1.5, of about 1.6, of about 1.7, of about 1.8, of about 1.9, of about 2.0, of about 2.1, of about 2.2, of about 2.3, of about 2.4, or of about 2.5. In some embodiments, the one or more organism from Table 1 having a Mean CRC Wald score less than zero in Column B of Table 1 has a Mean CRC Wald score in Column B of Table 1 of about −0.01, of about −0.02, of about −0.03, of about −0.04, of about −0.05, of about −0.06, of about −0.07, of about −0.08, of about −0.09, of about −0.1, of about −0.2, of about −0.3, of about −0.4, of about −0.5, of about −0.6, of about −0.7, of about −0.8, of about −0.9, of about −1.0, of about −1.1, of about −1.2, of about −1.3, of about −1.4, of about −1.5, of about −1.6, of about −1.7, of about −1.8, of about −1.9, of about −2.0, of about −2.1, of about −2.2, of about −2.3, of about −2.4, of about −2.5, of about −2.6, of about −2.7, or of about −2.8.

In some embodiments: (1) the one or more organism from Table 1 having a Mean CRC Wald score greater than zero in Column B of Table 1 has a Mean CRC Wald score in Column B of Table 1 of about 0.01, of about 0.02, of about 0.03, of about 0.04, of about 0.05, of about 0.06, of about 0.07, of about 0.08, of about 0.09, of about 0.1, of about 0.2, of about 0.3, of about 0.4, of about 0.5, of about 0.6, of about 0.7, of about 0.8, of about 0.9, of about 1.0, of about 1.1, of about 1.2, of about 1.3, of about 1.4, of about 1.5, of about 1.6, of about 1.7, of about 1.8, of about 1.9, of about 2.0, of about 2.1, of about 2.2, of about 2.3, of about 2.4, or of about 2.5; and (2) the one or more organism from Table 1 having a Mean CRC Wald score less than zero in Column B of Table 1 has a Mean CRC Wald score in Column B of Table 1 of about −0.01, of about −0.02, of about −0.03, of about −0.04, of about −0.05, of about −0.06, of about −0.07, of about −0.08, of about −0.09, of about −0.1, of about −0.2, of about −0.3, of about −0.4, of about −0.5, of about −0.6, of about −0.7, of about −0.8, of about −0.9, of about −1.0, of about −1.1, of about −1.2, of about −1.3, of about −1.4, of about −1.5, of about −1.6, of about −1.7, of about −1.8, of about −1.9, of about −2.0, of about −2.1, of about −2.2, of about −2.3, of about −2.4, of about −2.5, of about −2.6, of about −2.7, or of about −2.8.

### Certain Definitions

Prior to setting forth this disclosure in more detail, it may be helpful to an understanding thereof to provide additional definitions of certain terms to be used herein. Still more definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. “About” includes ±15%, ±10%, and ±5%. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter.

The terms “cancer” and “tumor” are used interchangeably herein and refer to proliferation or hyperproliferation of cells that results in dysregulated growth, unregulated growth, lack of differentiation, local tissue invasion, and/or metastasis.

As used herein, the terms “colorectal cancer” and “CRC” include colorectal adenomas and tumors.

As used herein, the terms “treatment,” “treat,” “treated,” or “treating” can include reversing, alleviating, and/or inhibiting the progression of or preventing or reducing the likelihood of the disease, disorder, or condition to which such term applies. When used with respect to a cancer, for example, the terms generally refer to reversing, alleviating, and/or inhibiting the progression of disease and/or symptoms.

As used herein, “subject” or “patient” refers to one or more individuals that are in need of receiving diagnosis, treatment, preventative measures, and/or therapy. Subjects that can be diagnosed or treated according to the present disclosure are, in general, human. However, additional subjects may include a non-human primate, cow, horse, sheep, goat, pig, dog, cat, mouse, rabbit, rat, or Guinea pig. The subjects can be male or female and can be any suitable age, including infant, juvenile, adolescent, adult, and geriatric subjects. In some embodiments, a subject is a human male. In some embodiments, a subject is a human female. In some embodiments, a subject is about 10, about 15, about 20, about 25, about 30, about 35, about 40, about 45, about 50, about 55, about 60, about 65, about 70, about 75, about 80, about 85, about 90, about 95, or about 100 years old. In some embodiments, a subject has a familial history of CRC, of polyps, of cancer, or any combination thereof. In some embodiments, a subject has been diagnosed with CRC or has previously had CRC. In some embodiments, a subject: is a smoker; does not engage in regular physical activity; has a diet that is low in fruit and/or vegetables (e.g., low relative to a typical recommended diet or relative to a typical recommended diet for that subject); has a low-fiber (e.g., low relative to a typical recommended diet or relative to a typical recommended diet for that subject), high-fat diet (e.g., high relative to a typical recommended diet or relative to a typical recommended diet for that subject); has a diet high in processed meats (e.g., high relative to a typical recommended diet or relative to a typical recommended diet for that subject); is overweight (e.g., clinically overweight as determined by a physician or according to a medically accepted standard); is obese (e.g., obese as determined by a physician or according to a medically accepted standard); consumes alcohol; uses tobacco; is over 30, 35, 40, 45, 50, 55, 60, 65, 70, 75, 80, 85, 90, 95, or 100 years of age; has or has had an inflammatory bowel disease (e.g., Crohn's disease, ulcerative colitis, or the like); has a genetic syndrome such as familial adenomatous polyposis (FAP) or hereditary non-polyposis colorectal cancer (Lynch syndrome); has a personal or family history of colorectal cancer or colorectal polyps; or any combination thereof.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), field programmable gate array (FPGA), a combination of hardware and software, and/or the like. The term “processor” as used herein means a single processor or multiple processors working independently or together to collectively perform a task.

Software may include one or more computer readable instructions that when executed by one or more components cause the component to perform a specified function. It should be understood that the algorithms described herein may be stored on one or more non-transitory memory. Exemplary non-transitory memory may include random access memory, read only memory, flash memory, and/or the like. Such non-transitory memory may be electrically based, optically based, and/or the like.

The term “healthcare provider” as used herein includes a person or group of persons capable of providing health services including, but not limited to, a Doctor of Medicine or osteopathy, podiatrist, dentist, chiropractor, clinical psychologist, optometrist, nurse practitioner, nurse-midwife, nurse, a clinical social worker, veterinarian, and the like. Further, “healthcare provider” may include any provider whom an insurance provider will accept medical codes to substantiate a claim for benefits.

As used herein, the terms “network-based”, “cloud-based”, and any variations thereof, may include the provision of configurable computational resources on demand via interfacing with a computer and/or computer network, with software and/or data at least partially located on a computer and/or computer network, by pooling processing power of two or more networked processors.

By way of background, standard intervention for individuals listed as high-risk for CRC is to implement colonoscopy earlier or intensify its use. Such intervention can be referred to as “screening” if the subject has no personal history of adenomatous polyps or CRC, and can be referred to as “surveillance” if the subject already has a history of adenomatous polyps or CRC. For example, if a subject has a first-degree relative who developed CRC at a young age, a recommendation would be to start screening earlier (10 years before the age at which the relative developed CRC), and if a subject was found to have adenomatous polyps on colonoscopy, then a recommendation would be for the follow-up colonoscopy to fall at a shorter interval (the length of which will typically depend on the level of concern about the polyp, which is based on size and histology).

Challenges with colonoscopy uptake include cost, access, and deployment. It is thought that colonoscopy alone will be insufficient for population-wide screening. There is a public health need and market for non-invasive screening (e.g. microbiome-based risk profiling), which could help by (1) identifying high-risk individuals for whom it would behoove society to pay for colonoscopy (e.g. transportation for someone who lives remotely, or procedural costs for someone who lacks insurance) to remove polyps and early CRCs, and (2) permit targeted preventative interventions.

Further, current preventative interventions (other than colonoscopy with polyp resection) include taking aspirin and lifestyle measures (e.g. a plant-based diet). However, the effect sizes of these interventions are small. As disclosed herein, microbiome manipulation may prevent polyps. As mentioned above, while several specific gut microbes have been identified as potentially carcinogenic, each appears to be causative in a small minority of CRC cases, and in those cases, estimated effect sizes are modest.

In the present disclosure, it was postulated that CRC risk may be shaped through cumulative effects of multiple diverse gut microbes, each of which may potentially have modest individual effect sizes, but which in time and in aggregate may result in adenomas and ultimately CRC.

A large-scale meta-analysis of global CRC cohorts was performed, pooling published metagenomic datasets to increase power. Hundreds of thousands of co-associated gut bacterial genes significantly enriched or depleted in CRC were identified that are widely encoded in genomes of diverse commensal organisms, including—unexpectedly—bacteria thought to be benign or beneficial.

Causality in gnotobiotic Apc^{Min/+} mice was tested using synthetic bacterial communities that had either a CRC-associated (“pro-tumor”) or a health-associated (“anti-tumor”) genomic make-up. It was found that the “pro-tumor” consortium induced significantly greater tumor burden than the “anti-tumor” consortium, providing in vivo validation of in silico predictions. Follow-up studies demonstrated that the pro-tumor consortium's tumorigenic effects were mediated via the tissue microenvironment rather than through direct intestinal epithelial cell growth promotion.

Thus, disclosed embodiments may be useful for microbiome-based colorectal cancer screening. This would enable wider deployment of screening than colonoscopy, among other advantages.

Table 1 shows a list of 357 genomes from the NCBI Representative Genomes collection with corresponding CRC Association scores, as described in the Example. Table 1 includes bacteria species that are health-associated (CRC Wald<0 in Column B) and CRC risk-associated (CRC Wald>0 in Column B). Column A lists organisms and Column B lists Mean CRC Wald scores.

TABLE 1
Health-associated and CRC-associated microbes

| | | | | | Column F | |
|---|---|---|---|---|---|---|
| | Column B | | Column D | Column E | (Unaligned | Column G |
| Column A | (Mean CRC | Column C | (CRC Wald >1 | (CRC Wald <1 | Genome | (Genome |
| (Organism Name) | Wald) | (Genome Assembly Accession) | (proportion)) | (proportion)) | (proportion)) | Size (bps)) |
| [Clostridium] | 1.692240586 | GCA_012317185.1_ASM1231718v1 | 0.532277793 | 0.023414941 | 0.444307266 | 4718910 |
| innocuum ATCC | | | | | | |
| 14501 | | | | | | |
| [Clostridium] | 0.059337397 | GCA_000144625.1_ASM14462v1 | 0.028689627 | 0.004237304 | 0.967073069 | 4662870 |
| saccharolyticum | | | | | | |
| WM1 WM1 | | | | | | |
| [Clostridium] | 0.097313936 | GCA_004295125.1_ASM429512v1 | 0.223001389 | 0.055437065 | 0.721561547 | 3658040 |
| scindens ATCC | | | | | | |
| 35704 ATCC 35704 | | | | | | |
| [Enterobacter] | 0.348042614 | GCA_001461805.1_ASM146180v1 | 0.250102383 | 0.010594202 | 0.739303416 | 4702950 |
| lignolyticus G5 | | | | | | |
| [Eubacterium] eligens | −1.487732822 | GCA_000146185.1_ASM14618v1 | 0.003378906 | 0.607578963 | 0.389042131 | 2831390 |
| ATCC 27750 ATCC | | | | | | |
| 27750 | | | | | | |
| [Ruminococcus] | −0.046547788 | GCA_009831375.1_ASM983137v1 | 0.021776518 | 0.077688994 | 0.900534488 | 3549190 |
| gnavus ATCC 29149 | | | | | | |
| ATCC 29149 | | | | | | |
| Acidaminococcus | 0.180377316 | GCA_000025305.1_ASM2530v1 | 0.108609434 | 0 | 0.891390566 | 2329770 |
| fermentans DSM | | | | | | |
| 20731 DSM 20731 | | | | | | |
| Adlercreutzia | −0.773289045 | GCA_000478885.1_ASM47888v1 | 0 | 0.459575271 | 0.540424729 | 2862530 |
| equolifaciens DSM | | | | | | |
| 19450 DSM 19450 | | | | | | |
| Akkermansia | 1.281897097 | GCA_009731575.1_ASM973157v1 | 0.674745176 | 0.003943775 | 0.321311048 | 2878460 |
| muciniphila JCM | | | | | | |
| 30893 | | | | | | |
| Alistipes communis | 0.529090434 | GCA_006542665.1_Acom_1.0 | 0.280301695 | 0.000774229 | 0.718924077 | 3301350 |
| 5CBH24 | | | | | | |
| Alistipes dispar | 1.252980032 | GCA_006542685.1_Adis_1.0 | 0.560335609 | 0.002011221 | 0.437653171 | 2962380 |
| 5CPEGH6 | | | | | | |
| Alistipes indistinctus | 0.68676223 | GCA_014163495.1_ASM1416349v1 | 0.22638827 | 0.000166689 | 0.773445041 | 3095580 |
| 2BBH45 | | | | | | |
| Alistipes megaguti | 0.306589017 | GCA_900604385.1_PRJEB28786 | 0.13676128 | 0.002106785 | 0.861131935 | 3270860 |
| Marseille-P5997 | | | | | | |
| Amedibacterium | 0.615762129 | GCA_010537335.1_ASM1053733v1 | 0.331680801 | 0.016859853 | 0.651459346 | 2488100 |
| intestinale 9CBEGH2 | | | | | | |
| Anaerostipes caccae | 0.283621561 | GCA_014131675.1_ASM1413167v1 | 0.240657862 | 0.042080975 | 0.717261162 | 3590720 |
| L1-92 DSM 14662 | | | | | | |
| Anaerostipes | 0.48782951 | GCA_005280655.1_ASM528065v1 | 0.351032083 | 0.022886655 | 0.626081263 | 3588860 |
| rhamnosivorans 1y2 | | | | | | |
| Anaerotignum | 0.032610632 | GCA_001561955.1_ASM156195v1 | 0.012111831 | 0.000316304 | 0.987571865 | 3120420 |
| propionicum DSM | | | | | | |
| 1682 X2 | | | | | | |
| Arsenophonus | 0.016557945 | GCA_013460135.1_ASM1346013v1 | 0.011530085 | 0.000603851 | 0.987866064 | 2424440 |
| endosymbiont of | | | | | | |
| Aphis craccivora Ash | | | | | | |
| Atlantibacter | 0.26020376 | GCA_008064855.1_ASM806485v1 | 0.173060399 | 0.003324435 | 0.823615166 | 4315320 |
| hermannii ATCC | | | | | | |
| 33651 | | | | | | |
| Bacteroides | 1.716042106 | GCA_001688725.2_ASM168872v2 | 0.488518636 | 0.002546318 | 0.508935047 | 4839930 |
| caecimuris I48 | | | | | | |
| Bacteroides | 1.689858017 | GCA_018292125.1_ASM1829212v1 | 0.658729871 | 0.000158024 | 0.341112106 | 7271070 |
| cellulosilyticus | | | | | | |
| CL06T03C01 | | | | | | |
| Bacteroides fragilis | 2.448614943 | GCA_016889925.1_ASM1688992v1 | 0.681628481 | 0.000383506 | 0.317988013 | 5248940 |
| FDAARGOS_1225 | | | | | | |
| Bacteroides | 0.64012954 | GCA_000186225.1_ASM18622v1 | 0.194658544 | 0.000952009 | 0.804389446 | 3998910 |
| helcogenes P 36-108 | | | | | | |
| P 36-108 | | | | | | |
| Bacteroides | 0.660880388 | GCA_002998535.1_ASM299853v1 | 0.186966955 | 0 | 0.813033045 | 3608980 |
| heparinolyticus F0111 | | | | | | |
| Bacteroides | 2.053437582 | GCA_009193295.2_ASM919329v2 | 0.574446753 | 0.003736921 | 0.421816326 | 5760090 |
| luhongzhouii HF-5141 | | | | | | |
| Bacteroides | 2.473990364 | GCA_014131755.1_ASM1413175v1 | 0.672818713 | 0.001973449 | 0.325207838 | 6304190 |
| thetaiotaomicron | | | | | | |
| DSM 2079 | | | | | | |
| Bacteroides uniformis | 2.476449179 | GCA_018292165.1_ASM1829216v1 | 0.702776536 | 0.001146914 | 0.29607655 | 4920160 |
| CL03T12C37 | | | | | | |
| CL03T12C37 | | | | | | |
| Bacteroides | 0.590486908 | GCA_002998435.1_ASM299843v1 | 0.172042275 | 0.000951279 | 0.827006446 | 3361790 |
| zoogleoformans | | | | | | |
| ATCC 33285 | | | | | | |
| Barnesiella | 0.431428127 | GCA_000512915.1_ASM51291v1 | 0.221143308 | 0.001549307 | 0.777307385 | 3076860 |
| viscericola DSM | | | | | | |
| 18177 C46, DSM 18177 | | | | | | |
| Bifidobacterium | −1.782988627 | GCA_003030905.1_ASM303090v1 | 0.002371341 | 0.682783487 | 0.314845172 | 2192430 |
| adolescentis 1-11 | | | | | | |
| Bifidobacterium | −0.645046612 | GCA_001025155.1_ASM102515v1 | 0.085848455 | 0.351033398 | 0.563118147 | 2021970 |
| angulatum DSM | | | | | | |
| 20098 = JCM 7096 | | | | | | |
| JCM 7096 | | | | | | |
| Bifidobacterium | −1.451827131 | GCA_000224965.2_ASM22496v2 | 0.000259984 | 0.684408175 | 0.315331841 | 1938580 |
| animalissubsp. lactis | | | | | | |
| BLC1 BLC1 | | | | | | |
| Bifidobacterium | −0.029803758 | GCA_000304215.1_ASM30421v1 | 0.000243621 | 0.01203156 | 0.987724819 | 2167300 |
| asteroides PRL2011 | | | | | | |
| PRL2011 | | | | | | |
| Bifidobacterium | −1.505524902 | GCA_000568975.1_ASM56897v1 | 0.002658022 | 0.576893033 | 0.420448945 | 2288920 |
| breve JCM 7017 JCM | | | | | | |
| 7017 | | | | | | |
| Bifidobacterium | −1.802925401 | GCA_001025195.1_ASM102519v1 | 0.004610679 | 0.676252212 | 0.319137109 | 2079520 |
| catenulatum DSM | | | | | | |
| 16992 = JCM 1194 = | | | | | | |
| LMG 11043 JCM 1194 | | | | | | |
| Bifidobacterium | −0.198122067 | GCA_002761235.1_ASM276123v1 | 0.000235238 | 0.084663025 | 0.915101737 | 2257290 |
| choerinum FMB-1 | | | | | | |
| Bifidobacterium | −0.026385291 | GCA_000737865.1_ASM73786v1 | 0.000300829 | 0.011064012 | 0.988635159 | 1755150 |
| coryneforme | | | | | | |
| LMG18911 | | | | | | |
| Bifidobacterium | −0.523195674 | GCA_001042595.1_ASM104259v1 | 0.003028831 | 0.221776247 | 0.775194922 | 2635670 |
| dentium JCM 1195 = | | | | | | |
| DSM 20436 JCM 1195 | | | | | | |
| Bifidobacterium | −0.173327421 | GCA_014898155.1_ASM1489815v1 | 0.000916175 | 0.066869462 | 0.932214363 | 2920840 |
| eulemuris DSM | | | | | | |
| 100216 | | | | | | |
| Bifidobacterium | −0.025668954 | GCA_000706765.1_ASM70676v1 | 0.000304402 | 0.011195411 | 0.988500187 | 1734550 |
| indicum LMG 11587 = | | | | | | |
| DSM 20214 LMG | | | | | | |
| 11587 | | | | | | |
| Bifidobacterium | −0.169799645 | GCA_014898175.1_ASM1489817v1 | 0.000535226 | 0.065482783 | 0.93398199 | 2965100 |
| lemurum DSM 28807 | | | | | | |
| Bifidobacterium | −1.779960633 | GCA_000196575.1_ASM19657v1 | 0.003005193 | 0.676187195 | 0.320807612 | 2408830 |
| longum subsp. | | | | | | |
| infantis 157F 157F | | | | | | |
| Bifidobacterium | −1.747946183 | GCA_003952825.1_ASM395282v1 | 0.006424496 | 0.678099699 | 0.315475805 | 2192390 |
| pseudocatenulatum 12 | | | | | | |
| Bifidobacterium | −0.294848953 | GCA_002282915.1_ASM228291v1 | 0 | 0.119931278 | 0.880068722 | 2008100 |
| pseudolongum UMB- | | | | | | |
| MBP-01 | | | | | | |
| Bifidobacterium | −0.266393545 | GCA_001042635.1_ASM104263v1 | 0.001921573 | 0.105193535 | 0.892884892 | 3158350 |
| scardovii JCM 12489 = | | | | | | |
| DSM 13734 JCM 12489 | | | | | | |
| Blautia argi KCTC 15426 | 0.064304115 | GCA_003287895.1_ASM328789v1 | 0.171927968 | 0.064203846 | 0.763868186 | 3297980 |
| Blautia hansenii DSM | −0.991424693 | GCA_002222595.2_ASM222259v2 | 0.071019749 | 0.44427339 | 0.484706861 | 3065950 |
| 20583 DSM 20583 | | | | | | |
| Blautia producta | −0.045376576 | GCA_014131715.1_ASM1413171v1 | 0.027897895 | 0.037617508 | 0.934484597 | 6245310 |
| DSM 2950 | | | | | | |
| Blautia | −0.054571161 | GCA_001689125.2_ASM168912v2 | 0.019335706 | 0.035663076 | 0.945001219 | 5128750 |
| pseudococcoides YL58 | | | | | | |
| Brenneria goodwinii | 0.036777253 | GCA_002291445.1_ASM229144v1 | 0.027835202 | 0.00098494 | 0.971179858 | 5360730 |
| FRB141 | | | | | | |
| Brenneria izadpanahii | 0.039240426 | GCA_017569925.1_ASM1756992v1 | 0.028685351 | 0.000799707 | 0.970514942 | 5330700 |
| Iran 50 | | | | | | |
| Brenneria nigrifluens | 0.043246937 | GCA_005484965.1_ASM548496v1 | 0.031410553 | 0.000597952 | 0.967991496 | 4891700 |
| DSM 30175 = ATCC | | | | | | |
| 13028 ATCC 13028 | | | | | | |
| Brenneria | 0.036981303 | GCA_005484945.1_ASM548494v1 | 0.028433823 | 0.000250987 | 0.971315189 | 4028090 |
| rubrifaciens 6D370 | | | | | | |
| Buttiauxella agrestis | 0.185371738 | GCA_013234275.1_ASM1323427v1 | 0.126133041 | 0.004393978 | 0.869472981 | 4566250 |
| DSM 9389 | | | | | | |
| Butyricimonas | 0.394568139 | GCA_003991565.1_ASM399156v1 | 0.130437744 | 0.001217743 | 0.868344513 | 4976420 |
| faecalis H184 | | | | | | |
| Butyricimonas virosa | 0.353905234 | GCA_016889065.1_ASM1688906v1 | 0.104604271 | 0.000509231 | 0.894886498 | 4813140 |
| FDAARGOS_1229 | | | | | | |
| Candidatus Doolittlea | 0.016037144 | GCA_900039485.1_DEMHIR | 0.011006873 | 0 | 0.988993127 | 846562 |
| endobia DEMHIR | | | | | | |
| Candidatus Sodalis | 0.020649152 | GCA_000517405.1_ASM51740v1 | 0.01494968 | 0.000385541 | 0.984664779 | 4513140 |
| pierantonius str. | | | | | | |
| SOPE SOPE | | | | | | |
| Cedecea lapagei | 0.176744 | GCA_900635955.1_36672_A01 | 0.120088146 | 0.002858464 | 0.87705339 | 4778440 |
| NCTC11466 | | | | | | |
| Cedecea neteri | 0.164283442 | GCA_002393445.1_ASM239344v1 | 0.112260984 | 0.004038725 | 0.883700291 | 5469300 |
| FDAARGOS_392 | | | | | | |
| Chania | 0.038722858 | GCA_000520015.2_ASM52001v2 | 0.030272331 | 0.000351483 | 0.969376187 | 5488180 |
| multitudinisentens | | | | | | |
| RB-25 RB-25 | | | | | | |
| Citrobacter | 0.478136961 | GCA_001558935.2_ASM155893v2 | 0.34617981 | 0.005545 | 0.648275191 | 5084040 |
| amalonaticus | | | | | | |
| FDAARGOS_165 | | | | | | |
| Citrobacter arsenatis | 0.348203638 | GCA_004353845.1_ASM435384v1 | 0.334664437 | 0.008175627 | 0.657159935 | 5370230 |
| LY-1 | | | | | | |
| Citrobacter braakii | 0.345097686 | GCA_009648935.1_ASM964893v1 | 0.343320881 | 0.008121216 | 0.648557903 | 4917490 |
| MiY-A | | | | | | |
| Citrobacter farmeri | 0.470733702 | GCA_019803045.1_ASM1980304v1 | 0.339949544 | 0.00662737 | 0.653423087 | 5406670 |
| CCRI-24236 | | | | | | |
| Citrobacter freundii | 0.384493915 | GCA_003812345.1_ASM381234v1 | 0.363761231 | 0.007556799 | 0.62868197 | 5102160 |
| FDAARGOS_549 | | | | | | |
| Citrobacter koseri | 0.471792461 | GCA_000018045.1_ASM1804v1 | 0.363611637 | 0.012086515 | 0.624301848 | 4735360 |
| ATCC BAA-895 | | | | | | |
| ATCC BAA-895 | | | | | | |
| Citrobacter pasteurii | 0.362715828 | GCA_019047765.1_ASM1904776v1 | 0.334882262 | 0.009456478 | 0.65566126 | 5021320 |
| FDAARGOS 1424 | | | | | | |
| Citrobacter portucalensis | 0.371233475 | GCA_008693605.1_ASM869360v1 | 0.355204956 | 0.00748092 | 0.637314123 | 4929340 |
| FDAARGOS_617 | | | | | | |
| Citrobacter rodentium | 0.426917347 | GCA_000027085.1_ASM2708v1 | 0.299485882 | 0.004640834 | 0.695873284 | 5444280 |
| ICC168 ICC168 | | | | | | |
| Citrobacter | 0.4174923 | GCA_009363175.1_ASM936317v1 | 0.310713417 | 0.009136848 | 0.680149735 | 5794230 |
| telavivensis 6105 | | | | | | |
| Citrobacter tructae | 0.392267749 | GCA_004684345.1_ASM468434v1 | 0.342308104 | 0.005202999 | 0.652488896 | 4946570 |
| SNU WT2 | | | | | | |
| Cloacibacillus | 0.768891176 | GCA_001701045.1_ASM170104v1 | 0.305267503 | 0.000528842 | 0.694203654 | 3585190 |
| porcorum CL-84 | | | | | | |
| Clostridioides | 0.056243252 | GCA_018885085.1_ASM1888508v1 | 0.020733223 | 0.005748934 | 0.973517843 | 4095890 |
| difficile S-0253 | | | | | | |
| Clostridium | 0.02877613 | GCA_001886875.1_ASM188687v1 | 0.010235112 | 0.003431317 | 0.986333571 | 4639910 |
| butyricum | | | | | | |
| CDC 51208 | | | | | | |
| Clostridium | −0.156443268 | GCA_000013285.1_ASM1328v1 | 0.001522716 | 0.064153064 | 0.93432422 | 3256680 |
| perfringens ATCC | | | | | | |
| 13124 ATCC 13124 | | | | | | |
| Collinsella | 0.010087873 | GCA_002736145.1_ASM273614v1 | 0.022492683 | 0.048282784 | 0.929224532 | 2306350 |
| aerofaciens indica | | | | | | |
| Coprobacter secundus | 0.22638847 | GCA_015097275.1_ASM1509727v1 | 0.081361966 | 0.000170444 | 0.918467591 | 4171470 |
| subsp. similis | | | | | | |
| 2CBH44 | | | | | | |
| Coprococcus comes | −2.862629839 | GCA_016904155.1_ASM1690415v1 | 0.006496159 | 0.59124655 | 0.402257291 | 3373070 |
| FDAARGOS_1339 | | | | | | |
| Cronobacter | 0.22774533 | GCA_001277255.1_ASM127725v1 | 0.152326936 | 0.00550997 | 0.842163094 | 4499480 |
| condimenti 1330 | | | | | | |
| LMG 26250 | | | | | | |
| Cronobacter | 0.21895395 | GCA_001277235.1_ASM127723v1 | 0.148537075 | 0.006193285 | 0.84526964 | 4628400 |
| dublinensis subsp. | | | | | | |
| dublinensis LMG | | | | | | |
| 23823 LMG 23823 | | | | | | |
| Cronobacter | 0.231288437 | GCA_001277215.2_ASM127721v2 | 0.160046583 | 0.007584895 | 0.832368522 | 4473760 |
| malonaticus LMG | | | | | | |
| 23826 LMG 23826 | | | | | | |
| Cronobacter | 0.224900784 | GCA_001277195.1_ASM127719v1 | 0.153251178 | 0.006646029 | 0.840102793 | 4364110 |
| muytjensii ATCC | | | | | | |
| 51329 ATCC 51329 | | | | | | |
| Cronobacter sakazakii | 0.229348064 | GCA_003516125.3_ASM351612v3 | 0.155879351 | 0.00583913 | 0.838281519 | 4437990 |
| CS-931 | | | | | | |
| Cronobacter | 0.225979353 | GCA_001277175.1_ASM127717v1 | 0.152533205 | 0.006355832 | 0.841110963 | 4436870 |
| universalis NCTC | | | | | | |
| 9529 NCTC 9529 | | | | | | |
| Desulfovibrio | 0.267227567 | GCA_001553605.1_ASM155360v1 | 0.079677832 | 0 | 0.920322168 | 3699310 |
| fairfieldensis CCUG | | | | | | |
| 45958 | | | | | | |
| Dialister massiliensis | −0.00146404 | GCA_900343095.1_PRJEB25867 | 0.010771731 | 0.001357618 | 0.987870651 | 2320240 |
| Marseille-P5638 | | | | | | |
| Dickeya aquatica 174/2 | 0.046038904 | GCA_900095885.1_Daq1742 | 0.032251486 | 0.000397862 | 0.967350652 | 4501560 |
| Dickeya chrysanthemi | 0.041493476 | GCA_000023565.1_ASM2356v1 | 0.030092545 | 0.000785857 | 0.969121597 | 4813850 |
| Ech1591 Ech1591 | | | | | | |
| Dickeya dadantii | 0.042192593 | GCA_000147055.1_ASM14705v1 | 0.030177948 | 0.000489965 | 0.969332087 | 4922800 |
| 3937 3937 | | | | | | |
| Dickeya dianthicola ME23 | 0.046769463 | GCA_003403135.1_ASM340313v1 | 0.033206154 | 0.000616004 | 0.966177843 | 4909060 |
| Dickeya fangzhongdai | 0.046091415 | GCA_002812485.1_ASM281248v1 | 0.032455166 | 0.000949637 | 0.966595197 | 5032450 |
| DSM 101947 | | | | | | |
| Dickeya paradisiaca | 0.040050929 | GCA_000023545.1_ASM2354v1 | 0.028883523 | 0.000720598 | 0.97039588 | 4679450 |
| Ech703 Ech703 | | | | | | |
| Dickeya poaceiphila | 0.045804403 | GCA_007858975.2_ASM785897v2 | 0.031819603 | 0.000378027 | 0.96780237 | 4317150 |
| NCPPB 569 | | | | | | |
| Dickeya solani PPO 9019 | 0.039219728 | GCA_002846995.1_ASM284699v1 | 0.028464885 | 0.000585802 | 0.970949313 | 4962430 |
| Dickeya zeae MS2 | 0.0456093 | GCA_002887555.1_ASM288755v1 | 0.032557252 | 0.000618348 | 0.966824401 | 4740050 |
| Dysosmobacter | 1.593323795 | GCA_005121165.2_ASM512116v2 | 0.346652927 | 0.01815716 | 0.635189913 | 3576110 |
| welbionis J115 | | | | | | |
| Edwardsiella anguillarum | 0.036019104 | GCA_000264765.2_ASM26476v2 | 0.030797178 | 0.001058053 | 0.968144769 | 4329650 |
| ET080813 ET080813 | | | | | | |
| Edwardsiella hoshinae | 0.038536244 | GCA_016026395.1_ASM1602639v1 | 0.034346927 | 0.001482273 | 0.964170799 | 3817110 |
| FDAARGOS_940 | | | | | | |
| Edwardsiella ictaluri | 0.036411057 | GCA_000022885.2_ASM2288v2 | 0.030421006 | 0.000884505 | 0.968694489 | 3812300 |
| 93-146 93-146 | | | | | | |
| Edwardsiella tarda | 0.035649971 | GCA_002504285.1_ASM250428v1 | 0.032929947 | 0.002286186 | 0.964783867 | 3720170 |
| KC-Pc-HB1 | | | | | | |
| Eggerthella | −0.014002834 | GCA_009834925.2_ASM983492v2 | 0.001175518 | 0.015725789 | 0.983098693 | 4175180 |
| guodeyinii HF-1101 | | | | | | |
| Eggerthella lenta C592 | −0.036216093 | GCA_002148255.1_ASM214825v1 | 0.010443894 | 0.03652232 | 0.953033786 | 3593200 |
| Enterobacter asburiae | 0.17161444 | GCA_007035645.1_ASM703564v1 | 0.279934475 | 0.2533278 | 0.466737725 | 4770720 |
| 1808-013 | | | | | | |
| Enterobacter bugandensis | 0.216054792 | GCA_015137655.1_ASM1513765v1 | 0.300696975 | 0.245840479 | 0.453462547 | 4635750 |
| STN0717-56 | | | | | | |
| Enterobacter chengduensis | 0.188054976 | GCA_001984825.2_ASM198482v2 | 0.271679455 | 0.22131419 | 0.507006355 | 5218120 |
| WCHECl-C4 = | | | | | | |
| WCHECh050004 | | | | | | |
| Enterobacter cloacae | 0.249015012 | GCA_000770155.1_ASM77015v1 | 0.291982676 | 0.195864501 | 0.512152823 | 4848750 |
| GGT036 | | | | | | |
| Enterobacter kobei | 0.299304617 | GCA_018323985.1_ASM1832398v1 | 0.215808737 | 0.014503638 | 0.769687625 | 4737570 |
| JCM 8580 | | | | | | |
| Enterobacter ludwigii | 0.23386223 | GCA_001750725.1_ASM175072v1 | 0.278430858 | 0.191813874 | 0.529755268 | 4952770 |
| EN-119 | | | | | | |
| Enterobacter | 0.307263741 | GCA_009176645.1_ASM917664v1 | 0.314421692 | 0.183033514 | 0.502544794 | 4476590 |
| oligotrophicus CCA6 | | | | | | |
| Enterobacter | 0.165427244 | GCA_001729805.1_ASM172980v1 | 0.287192449 | 0.269321633 | 0.443485918 | 4900000 |
| roggenkampii DSM 16690 | | | | | | |
| Enterobacter sichuanensis | 0.201936254 | GCA_009036245.1_ASM903624v1 | 0.286712838 | 0.234912202 | 0.478374959 | 4711390 |
| SGAir0282 | | | | | | |
| Enterobacter soli LF7a | 0.253827241 | GCA_000224675.1_ASM22467v1 | 0.270244986 | 0.15590258 | 0.573852434 | 5012130 |
| Enterocloster bolteae | 1.851028749 | GCA_002234575.2_ASM223457v2 | 0.56221175 | 0.020405229 | 0.417383022 | 6614040 |
| ATCC BAA-613 | | | | | | |
| Enterococcus | −0.00780812 | GCA_000157355.2_ASM15735v2 | 0.001603604 | 0.010991515 | 0.987404881 | 3427280 |
| casseliflavus EC20 EC20 | | | | | | |
| Enterococcus | −0.016619634 | GCA_900474605.1_41594_C01 | 0.001184341 | 0.015755699 | 0.98305996 | 2421600 |
| cecorum NCTC12421 | | | | | | |
| Enterococcus faecium SRR24 | −0.012482993 | GCA_009734005.2_ASM973400v2 | 0.014316594 | 0.020380926 | 0.96530248 | 2919200 |
| Enterococcus hirae R17 | −0.004689365 | GCA_001641305.1_ASM164130v1 | 0.005648196 | 0.01032952 | 0.984022283 | 2960060 |
| Enterococcus lactis CX 2-6_2 | −0.013667364 | GCA_019343125.1_ASM1934312v1 | 0.03502806 | 0.016328027 | 0.948643913 | 2728070 |
| Enterococcus | −0.024585099 | GCA_011397115.1_ASM1139711v1 | 0.008779644 | 0.027310114 | 0.963910242 | 2844990 |
| saigonensis VE80 | | | | | | |
| Enterococcus | −0.00681069 | GCA_002290025.1_ASM229002v1 | 0.004338592 | 0.012711951 | 0.982949457 | 2646250 |
| thailandicus a523 | | | | | | |
| Enterococcus | −0.02718728 | GCA_002197645.1_ASM219764v1 | 0 | 0.028299667 | 0.971700333 | 4155950 |
| wangshanyuanii MN05 | | | | | | |
| Erwinia amylovora | 0.053414308 | GCA_000091565.1_ASM9156v1 | 0.036271561 | 0.000834935 | 0.962893503 | 3833830 |
| CFBP1430 CFPB1430 | | | | | | |
| Erwinia billingiae | 0.047848121 | GCA_000196615.1_ASM19661v1 | 0.033105559 | 0.00094541 | 0.965949031 | 5372270 |
| Eb661 Eb661 | | | | | | |
| Erwinia gerundensis | 0.055104343 | GCA_001517405.1_EM595 | 0.037056319 | 0.000812718 | 0.962130963 | 4481260 |
| E_g_EM595 | | | | | | |
| Erwinia persicina Cp2 | 0.054465927 | GCA_019844095.1_ASM1984409v1 | 0.038690133 | 0.000885709 | 0.960424158 | 4802930 |
| Erwinia pyrifoliae EpK1/15 | 0.052026701 | GCA_002952315.1_ASM295231v1 | 0.034775056 | 0.00078539 | 0.964439554 | 4075680 |
| Erwinia tasmaniensis | 0.054156041 | GCA_000026185.1_ASM2618v1 | 0.037069122 | 0.001119508 | 0.96181137 | 4067860 |
| Et1/99 Et1/99 | | | | | | |
| Erysipelatoclostridium | 0.011663287 | GCA_016728785.1_ASM1672878v1 | 0.002771664 | 0.021757645 | 0.975470692 | 3543720 |
| ramosum FDAARGOS_1105 | | | | | | |
| Escherichia albertii | 0.937807165 | GCA_016904755.2_ASM1690475v2 | 0.589493296 | 0.002428809 | 0.408077895 | 4631900 |
| Sample 167 | | | | | | |
| Escherichia | 0.787705832 | GCA_013892435.1_ASM1389243v1 | 0.50968849 | 0.004430613 | 0.485880897 | 4784440 |
| fergusonii RHB 19-C05 | | | | | | |
| Escherichia marmotae | 1.022907064 | GCA_900637015.1_46514_C01 | 0.649795521 | 0.004058791 | 0.346145688 | 4450340 |
| NCTC11133 | | | | | | |
| Eubacterium | 0.640179441 | GCA_000152245.2_ASM15224v2 | 0.220677785 | 0.028614848 | 0.750707367 | 4316710 |
| callanderi KIST612 | | | | | | |
| Eubacterium limosum | 0.651722381 | GCA_000807675.2_ASM80767v2 | 0.216759367 | 0.022521502 | 0.760719131 | 4422840 |
| ATCC 8486 | | | | | | |
| Eubacterium | 0.791927708 | GCA_002441855.2_ASM244185v2 | 0.267400115 | 0.022585591 | 0.710014294 | 4337500 |
| maltosivorans YI | | | | | | |
| Faecalibacillus | −2.303458237 | GCA_015097455.1_ASM1509745v1 | 0.000977359 | 0.590945582 | 0.40807706 | 2869980 |
| intestinalis 14EGH31 | | | | | | |
| Faecalibacterium | −1.084196012 | GCA_003312465.1_ASM331246v1 | 0.013252371 | 0.478723232 | 0.508024396 | 2970940 |
| prausnitzii APC918/95b | | | | | | |
| Filifactor alocis ATCC | 0.039612675 | GCA_000163895.2_ASM16389v2 | 0.018455109 | 0.000256343 | 0.981288548 | 1931010 |
| 35896 ATCC 35896 | | | | | | |
| Flavonifractor plautii | 1.688682247 | GCA_010508875.1_ASM1050887v1 | 0.48487074 | 0.00349351 | 0.51163575 | 3985390 |
| JCM 32125 | | | | | | |
| Fusobacterium | 0.787214165 | GCA_016724785.1_ASM1672478v1 | 0.222426899 | 0.000427256 | 0.777145845 | 2352220 |
| canifelinum | | | | | | |
| FDAARGOS_1126 | | | | | | |
| Fusobacterium | 0.841019322 | GCA_003019695.1_ASM301969v1 | 0.389532903 | 0 | 0.610467097 | 1678880 |
| gonidiaformans | | | | | | |
| ATCC 25563 ATCC 25563 | | | | | | |
| Fusobacterium hwasookii | 0.717913296 | GCA_001455085.1_ASM145508v1 | 0.204625348 | 0.000413492 | 0.794961161 | 2430520 |
| ChDC F206 ChDC F206 | | | | | | |
| Fusobacterium nucleatum | 0.806717495 | GCA_001457555.1_NCTC10562 | 0.227636392 | 0 | 0.772363608 | 2455060 |
| subsp. polymorphum | | | | | | |
| NCTC10562 | | | | | | |
| Fusobacterium | 0.785731398 | GCA_002763625.1_ASM276362v1 | 0.226443815 | 0 | 0.773556185 | 2372880 |
| pseudoperiodonticum | | | | | | |
| KCOM 1261 | | | | | | |
| Fusobacterium | 1.315836733 | GCA_003019675.1_ASM301967v1 | 0.472097533 | 0 | 0.527902467 | 3537680 |
| ulcerans ATCC 49185 | | | | | | |
| Fusobacterium | 1.619378689 | GCA_003019655.1_ASM301965v1 | 0.517908476 | 0 | 0.482091524 | 3346460 |
| varium ATCC 27725 | | | | | | |
| ATCC 27725 | | | | | | |
| Gemella morbillorum | 1.541454318 | GCA_009730315.1_ASM973031v1 | 0.412777544 | 0.003147602 | 0.584074855 | 1779450 |
| FDAARGOS_741 | | | | | | |
| Gibbsiella | 0.036373474 | GCA_002291425.1_ASM229142v1 | 0.030327601 | 0.000918084 | 0.968754314 | 5548510 |
| quercinecans FRB97 | | | | | | |
| Hafnia alvei A23BA | 0.035247836 | GCA_011617105.1_ASM1161710v1 | 0.050130028 | 0.000341991 | 0.949527981 | 4772050 |
| Jejubacter calystegiae | 0.126100467 | GCA_005671395.1_ASM567139v1 | 0.083655939 | 0.001672262 | 0.914671799 | 5182800 |
| KSNA2 | | | | | | |
| Jinshanibacter | 0.021169594 | GCA_004295645.1_ASM429564v1 | 0.014598635 | 0.000218267 | 0.985183098 | 4631940 |
| zhutongyuii CF-458 | | | | | | |
| Klebsiella aerogenes | 0.538886432 | GCA_007632255.1_ASM763225v1 | 0.412296186 | 0.008190853 | 0.579512961 | 5249270 |
| Ka37751 | | | | | | |
| Klebsiella huaxiensis | 0.618519715 | GCA_003261575.2_ASM326157v2 | 0.451726201 | 0.002901522 | 0.545372276 | 6300830 |
| WCHK1090001 | | | | | | |
| Klebsiella | 0.744292238 | GCA_015139575.1_ASM1513957v1 | 0.566154019 | 0.007461469 | 0.426384512 | 6041840 |
| michiganensis THO-011 | | | | | | |
| Klebsiella oxytoca | 0.814380195 | GCA_002984395.1_ASM298439v1 | 0.5770694 | 0.002017746 | 0.420912854 | 6049820 |
| FDAARGOS_335 | | | | | | |
| Klebsiella | 0.735286243 | GCA_016415705.1_ASM1641570v1 | 0.601592063 | 0.006405534 | 0.392002404 | 5391120 |
| quasipneumoniae KqPF26 | | | | | | |
| Klebsiella variicola FH-1 | 0.739795657 | GCA_013305245.1_ASM1330524v1 | 0.617327269 | 0.006461834 | 0.376210897 | 5652420 |
| Kluyvera ascorbata TP1631 | 0.325942353 | GCA_015099135.1_ASM1509913v1 | 0.23874902 | 0.012910258 | 0.748340721 | 5371310 |
| Kosakonia arachidis | 0.256049276 | GCA_009363135.1_ASM936313v1 | 0.181005175 | 0.011837934 | 0.807156891 | 5176410 |
| KACC 18508 | | | | | | |
| Kosakonia cowanii FBS 223 | 0.272467031 | GCA_004089895.1_ASM408989v1 | 0.186001921 | 0.008507042 | 0.805491037 | 4686000 |
| Kosakonia oryzae Ola 51 | 0.264330359 | GCA_001658025.2_ASM165802v2 | 0.1860222 | 0.014357035 | 0.799620765 | 5416160 |
| Kosakonia pseudosacchari | 0.280895522 | GCA_015167415.1_ASM1516741v1 | 0.19844295 | 0.015583294 | 0.785973756 | 5003050 |
| BDA62-3 | | | | | | |
| Kosakonia radicincitans | 0.255981249 | GCA_008330085.1_ASM833008v1 | 0.182302234 | 0.014266097 | 0.80343167 | 5774740 |
| DSM 107547 | | | | | | |
| Kosakonia sacchari BO-1 | 0.290436212 | GCA_001683395.1_ASM168339v1 | 0.204699813 | 0.01635153 | 0.778948657 | 4902110 |
| Lachnoclostridium | −0.072355841 | GCA_900120345.1_PRJEB18024 | 0.037119189 | 0.058970792 | 0.903910019 | 3500750 |
| phocaeense Marseille-P3177 | | | | | | |
| Lactiplantibacillus | −0.476355373 | GCA_003641145.1_ASM364114v1 | 0.001109683 | 0.156032453 | 0.842857864 | 3368530 |
| paraplantarum DSM 10667 | | | | | | |
| Lactiplantibacillus | −0.326349267 | GCA_003641185.1_ASM364118v1 | 0.000456778 | 0.106412865 | 0.893130357 | 3671370 |
| pentosus DSM 20314 | | | | | | |
| Lactiplantibacillus | −0.641489296 | GCA_003269405.1_ASM326940v1 | 0.00117354 | 0.205026847 | 0.793799613 | 3231250 |
| plantarum SK151 | | | | | | |
| Lactobacillus | −0.022945731 | GCA_008831485.1_ASM883148v1 | 0.002289328 | 0.01433458 | 0.983376091 | 1683900 |
| acetotolerans LA749 | | | | | | |
| Lactobacillus | −0.190573228 | GCA_000389675.2_ASM38967v2 | 0.003753804 | 0.111588287 | 0.88465791 | 1991580 |
| acidophilus La-14 La-14 | | | | | | |
| Lactobacillus amylolyticus L5 | −0.072954605 | GCA_003999355.1_ASM399935v1 | 0.007011036 | 0.043964177 | 0.949024788 | 1601190 |
| Lactobacillus amylovorus | −0.759037318 | GCA_000194115.1_ASM19411v1 | 0.006242508 | 0.360346266 | 0.633411226 | 1977090 |
| GRL1118 GRL1118 | | | | | | |
| Lactobacillus delbrueckii | −0.657717279 | GCA_003351805.1_ASM335180v1 | 0.004645827 | 0.538253675 | 0.457100497 | 1848110 |
| subsp. bulgaricus L99 | | | | | | |
| Lactobacillus gasseri HL20 | 0.311173496 | GCA_017638885.1_ASM1763888v1 | 0.385999558 | 0.010723551 | 0.603276892 | 1989080 |
| Lactobacillus johnsonii GHZ10a | 0.082984167 | GCA_014841035.1_ASM1484103v1 | 0.242378289 | 0.007435876 | 0.750185835 | 2015230 |
| Lactobacillus | −0.145001888 | GCA_014656585.1_ASM1465658v1 | 0.007113446 | 0.087319829 | 0.905566725 | 2173630 |
| kefiranofaciens 1207 | | | | | | |
| Lactobacillus | 0.35666423 | GCA_005886075.1_ASM588607v1 | 0.431092449 | 0.017942669 | 0.550964882 | 2030300 |
| paragasseri JV-V03 JV-V03 | | | | | | |
| Lactobacillus | 0.147621367 | GCA_017894345.1_ASM1789434v1 | 0.24072173 | 0.005167601 | 0.754110669 | 2041760 |
| taiwanensis CLG01 | | | | | | |
| Lactobacillus | −0.225949225 | GCA_016647595.1_ASM1664759v1 | 0.004846443 | 0.12712797 | 0.868025588 | 2246390 |
| ultunensis Kx293C1 | | | | | | |
| Lactococcus | −0.072324378 | GCA_003627095.1_ASM362709v1 | 0.001354041 | 0.061084103 | 0.937561856 | 2758410 |
| allomyrinae 1JSPR-7 | | | | | | |
| Lactococcus garvieae | −0.032322826 | GCA_016026695.1_ASM1602669v1 | 0.000246121 | 0.024081004 | 0.975672875 | 2084340 |
| FDAARGOS 929 | | | | | | |
| Lactococcus lactis | −0.36114273 | GCA_000468955.1_ASM46895v1 | 0.00382316 | 0.312914443 | 0.683262397 | 2427050 |
| subsp. cremoris KW2 KW2 | | | | | | |
| Lactococcus lactis | −0.551438678 | GCA_000344575.1_ASM34457v1 | 0.004409305 | 0.473146064 | 0.522444631 | 2421470 |
| subsp. lactis IO-1 IO-1 | | | | | | |
| Lactococcus raffinolactis | −0.018695456 | GCA_002310475.1_ASM231047v1 | 0.000366455 | 0.011311692 | 0.988321853 | 2292230 |
| WiKim0068 | | | | | | |
| Lactococcus | −0.189357268 | GCA_017068355.1_ASM1706835v1 | 0.001888627 | 0.161273119 | 0.836838254 | 1995100 |
| taiwanensisK_LL004 | | | | | | |
| Lancefieldella parvulum | −0.017463275 | GCA_000024225.1_ASM2422v1 | 0.001261166 | 0.011088152 | 0.987650682 | 1543810 |
| DSM 20469 DSM 20469 | | | | | | |
| Leclercia adecarboxylata | 0.29324486 | GCA_001518835.1_ASM151883v1 | 0.243743443 | 0.07618778 | 0.680068777 | 4803920 |
| USDA-ARS-USMARC-60222 | | | | | | |
| Leminorella richardii | 0.02914081 | GCA_900478135.1_28193_H01 | 0.020125645 | 0.000513044 | 0.979361311 | 3976270 |
| NCTC12151 | | | | | | |
| Ligilactobacillus | 0.165832092 | GCA_009933595.1_ASM993359v1 | 0.136105182 | 0.00244652 | 0.861448298 | 1906790 |
| animalis P38 | | | | | | |
| Ligilactobacillus | 0.12936711 | GCA_003288115.1_ASM328811v1 | 0.106282608 | 0.000512126 | 0.893205265 | 2290450 |
| murinus CR147 | | | | | | |
| Ligilactobacillus | −0.489453951 | GCA_001011095.1_ASM101109v1 | 0.027826078 | 0.435630522 | 0.5365434 | 1978360 |
| salivarius str. Ren Ren | | | | | | |
| Limnobaculum | 0.020910563 | GCA_003096015.2_ASM309601v2 | 0.014683076 | 0.00026316 | 0.985053764 | 3841770 |
| parvum HYN0051 | | | | | | |
| Limosilactobacillus | −0.002951053 | GCA_008876665.1_ASM887666v1 | 0.026968561 | 0.001940751 | 0.971090688 | 1752930 |
| frumenti LF145 | | | | | | |
| Limosilactobacillus | −0.005939139 | GCA_009428965.1_ASM942896v1 | 0.01058451 | 0.00116867 | 0.98824682 | 1714770 |
| pontis LP475 | | | | | | |
| Limosilactobacillus | 0.1363188 | GCA_009362935.1_ASM936293v1 | 0.175863325 | 0.003879222 | 0.820257454 | 1894710 |
| vaginalis LV515 | | | | | | |
| Longicatena | 0.437903008 | GCA_018406465.1_ASM1840646v1 | 0.290715133 | 0.022889334 | 0.686395533 | 3103760 |
| caecimuris 3BBH23 | | | | | | |
| Lonsdalea britannica 477 | 0.032368286 | GCA_003515985.1_ASM351598v1 | 0.025077635 | 0.000741115 | 0.974181249 | 4015570 |
| Lonsdalea populi N-5-1 | 0.039835429 | GCA_015999465.1_ASM1599946v1 | 0.028197455 | 0.000389407 | 0.971413137 | 3859710 |
| Mageeibacillus indolicus | 0.012055161 | GCA_000025225.2_ASM2522v1 | 0.010526316 | 0.00112557 | 0.988348114 | 1809750 |
| UPII9-5 UPII9-5 | | | | | | |
| Massilistercora timonensis | −0.298605438 | GCA_900312975.1_PRJEB24953 | 0.038573579 | 0.139016966 | 0.822409454 | 2769590 |
| Marseille-P3756 | | | | | | |
| Megasphaera elsdenii | 0.001617385 | GCA_001304715.1_ASM130471v1 | 0.035085751 | 0.002317967 | 0.962596282 | 2504350 |
| 14-14 14-14 | | | | | | |
| Megasphaera | 0.108145793 | GCA_003367905.1_ASM336790v1 | 0.107368552 | 0.001515403 | 0.891116045 | 2652760 |
| stantonii AJH120 | | | | | | |
| Mixta gaviniae DSM 22758 | 0.065680509 | GCA_002953195.1_ASM295319v1 | 0.047426346 | 0.00124039 | 0.951333264 | 4527610 |
| Mixta intestinalis | 0.073985252 | GCA_009914055.1_ASM991405v1 | 0.052250403 | 0.002608194 | 0.945141403 | 4784920 |
| SRCM103226 | | | | | | |
| Morganella morganii L241 | 0.023742833 | GCA_003955965.1_ASM395596v1 | 0.017748504 | 0.000259456 | 0.981992039 | 3896610 |
| Murdochiella vaginalis | 0.054705765 | GCA_900119705.1_PRJEB14245 | 0.025154204 | 0.005949781 | 0.968896015 | 1671490 |
| Marseille-P2341 | | | | | | |
| Muribaculum | 0.017280681 | GCA_001688845.2_ASM168884v2 | 0.017744355 | 0.002885261 | 0.979370384 | 3306460 |
| intestinale YL27 | | | | | | |
| Ornithobacterium | 0.037806178 | GCA_000756505.1_ASM75650v1 | 0.010195298 | 0 | 0.989804702 | 2397870 |
| rhinotracheale ORT- | | | | | | |
| UMN 88 ORT-UMN 88 | | | | | | |
| Paeniclostridium | −0.034538649 | GCA_002865995.1_ASM286599v1 | 0.000380773 | 0.013815795 | 0.985803432 | 3584810 |
| sordellii AM370 | | | | | | |
| Pantoea agglomerans | 0.053952786 | GCA_019048385.1_ASM1904838v1 | 0.037837435 | 0.000785802 | 0.961376763 | 4692020 |
| FDAARGOS 1447 | | | | | | |
| Pantoea alhagi LTYR-11Z | 0.065171481 | GCA_002101395.1_ASM210139v1 | 0.045100433 | 0.0014012 | 0.953498367 | 4316300 |
| Pantoea ananatis | 0.054039626 | GCA_000233595.1_ASM23359v1 | 0.037665113 | 0.001076199 | 0.961258688 | 4867130 |
| PA13 PA13 | | | | | | |
| Pantoea dispersa Lsch | 0.05902765 | GCA_019890955.1_ASM1989095v1 | 0.041594781 | 0.001656889 | 0.956748331 | 4885060 |
| Pantoea eucalypti LMG 24197 | 0.056523789 | GCA_009646115.1_ASM964611v1 | 0.038626253 | 0.001349659 | 0.960024088 | 4798990 |
| Pantoea stewartii ZJ-FGZX1 | 0.053564616 | GCA_011044475.1_ASM1104447v1 | 0.037481487 | 0.001066857 | 0.961451656 | 4982860 |
| Pantoea vagans LMG 24199 | 0.061085126 | GCA_004792415.1_ASM479241v1 | 0.042282056 | 0.001262543 | 0.956455401 | 4790330 |
| Parabacteroides | 0.67388244 | GCA_017873595.1_ASM1787359v1 | 0.264079432 | 0.001364558 | 0.73455601 | 6881350 |
| goldsteinii MTS01 | | | | | | |
| Paraclostridium | −0.033826895 | GCA_019916025.1_ASM1991602v1 | 0.000738597 | 0.01311052 | 0.986150882 | 3566220 |
| bifermentans DSM 14991 | | | | | | |
| Paraprevotella | 0.714558425 | GCA_900683745.1_Para- | 0.439537539 | 0.000402878 | 0.560059583 | 4125320 |
| xylaniphila YIT | | prevotella_xylaniphila_82A6 | | | | |
| 11841 Paraprevotella | | | | | | |
| xylaniphila 82A6 | | | | | | |
| Parolsenella catena | 0.00188688 | GCA_003966955.1_ASM396695v1 | 0 | 0.02191363 | 0.97808637 | 1796690 |
| JCM 31932 | | | | | | |
| Parvimonas micra | 2.063330704 | GCA_003454775.1_ASM345477v1 | 0.57032241 | 0.000714862 | 0.428962729 | 1661860 |
| KCOM 1037 | | | | | | |
| Pectobacterium | 0.039556044 | GCA_015689195.1_ASM1568919v1 | 0.030551853 | 0.000620909 | 0.968827238 | 4995900 |
| aroidearum L6 | | | | | | |
| Pectobacterium | 0.037817445 | GCA_000740965.1_ASM74096v1 | 0.028490222 | 0.000479475 | 0.971030303 | 5024250 |
| atrosepticum 21A | | | | | | |
| Pectobacterium | 0.041896359 | GCA_009873295.1_ASM987329v1 | 0.03249189 | 0.001898194 | 0.965609916 | 4851980 |
| brasiliense 1692 | | | | | | |
| Pectobacterium | 0.041885129 | GCA_013488025.1_ASM1348802v1 | 0.030809939 | 0.000431706 | 0.968758355 | 4892220 |
| carotovorum WPP14 | | | | | | |
| Pectobacterium | 0.040680247 | GCA_009931295.1_ASM993129v1 | 0.030212577 | 0.000878191 | 0.968909232 | 5100260 |
| odoriferum JK2.1 | | | | | | |
| Pectobacterium | 0.033029063 | GCA_003992745.1_ASM399274v1 | 0.027498326 | 0.001873816 | 0.970627858 | 5227300 |
| parmentieri IFB5427 | | | | | | |
| Pectobacterium | 0.037646493 | GCA_002288545.1_ASM228854v1 | 0.02852756 | 0.000471406 | 0.971001034 | 5008420 |
| polaris NIBIO1392 | | | | | | |
| Pectobacterium | 0.042271517 | GCA_012427845.1_ASM1242784v1 | 0.03132747 | 0.00060954 | 0.96806299 | 4793780 |
| punjabense SS95 | | | | | | |
| Pectobacterium wasabiae | 0.034904775 | GCA_001742185.1_ASM174218v1 | 0.027237901 | 0.001212318 | 0.971549781 | 5043230 |
| CFBP 3304 CFBP 3304 | | | | | | |
| Phascolarctobacterium | 1.414952206 | GCA_003945365.1_PFJ30894_01 | 0.670192351 | 0.004194967 | 0.325612683 | 2454370 |
| faecium JCM 30894 | | | | | | |
| Phocaeicola coprophilus | 1.040522408 | GCA_016888945.1_ASM1688894v1 | 0.575032149 | 0.001652563 | 0.423315288 | 4113610 |
| FDAARGOS_1220 | | | | | | |
| Phocaeicola salanitronis | 0.276766451 | GCA_000190575.1_ASM19057v1 | 0.140900419 | 0.002989793 | 0.856109788 | 4308660 |
| DSM 18170 DSM 18170 | | | | | | |
| Phocaeicola vulgatus | 2.551015425 | GCA_018289355.1_ASM1828935v1 | 0.690201902 | 0.0015701 | 0.308227997 | 5306030 |
| CL06T03C24 | | | | | | |
| Phoenicibacter congonensis | 0.011998205 | GCA_900169485.1_PRJEB19959 | 0.003839194 | 0.011946463 | 0.984214343 | 1447960 |
| Marseille-P3241 | | | | | | |
| Photorhabdus akhurstii | 0.018106676 | GCA_019090985.1_ASM1909098v1 | 0.011980553 | 0.000176554 | 0.987842893 | 5726280 |
| 0813-124 phase II | | | | | | |
| Photorhabdus asymbiotica | 0.026492559 | GCA_000196475.1_ASM19647v1 | 0.016560204 | 0.000198463 | 0.983241332 | 5094140 |
| ATCC43949 | | | | | | |
| Photorhabdus laumondii | 0.017635068 | GCA_000196155.1_ASM19615v1 | 0.011813872 | 0.000402356 | 0.987783772 | 5688990 |
| subsp. laumondii TTO1 TTO1 | | | | | | |
| Photorhabdus thracensis | 0.020559847 | GCA_001010285.1_ASM101028v1 | 0.014255989 | 0.000414991 | 0.98532902 | 5147100 |
| DSM 15199 | | | | | | |
| Phytobacter | 0.272324827 | GCA_012923785.1_ASM1292378v1 | 0.194009668 | 0.0140088 | 0.791981531 | 5527240 |
| diazotrophicus UAEU22 | | | | | | |
| Phytobacter ursingii | 0.265591389 | GCA_001022135.1_ASM102213v1 | 0.18917789 | 0.012266215 | 0.798555895 | 6166450 |
| CAV1151 | | | | | | |
| Pluralibacter gergoviae | 0.238787947 | GCA_003019925.1_ASM301992v1 | 0.166264552 | 0.003169147 | 0.830566301 | 5408080 |
| FDAARGOS_186 | | | | | | |
| Porphyromonas asaccharolytica | 1.278140215 | GCA_000212375.1_ASM21237v1 | 0.457275758 | 0 | 0.542724242 | 2186370 |
| DSM 20707 DSM 20707 | | | | | | |
| Porphyromonas cangingivalis | 0.042270426 | GCA_900638305.1_57043_C01 | 0.012106734 | 0 | 0.987893266 | 2404860 |
| NCTC12856 | | | | | | |
| Porphyromonas crevioricanis | 0.064841131 | GCA_900476255.1_53750_A02 | 0.019777345 | 0 | 0.980222655 | 2133350 |
| NCTC12858 | | | | | | |
| Porphyromonas gingivalis | 0.192901722 | GCA_000010505.1_ASM1050v1 | 0.075333455 | O | 0.924666545 | 2354890 |
| ATCC 33277 ATCC 33277 | | | | | | |
| Pragia fontium | 0.02003672 | GCA_900638655.1_58635_F02 | 0.014232302 | 0.000250328 | 0.985517369 | 4038700 |
| NCTC12284 | | | | | | |
| Prevotella dentalis | 0.041523169 | GCA_000242335.3_ASM24233v3 | 0.026349095 | 0.000303563 | 0.973347342 | 3350210 |
| DSM 3688 DSM 3688 | | | | | | |
| Prevotella denticola F0115 | 0.123816901 | GCA_018128205.1_ASM1812820v1 | 0.050206017 | 0.000182518 | 0.949611465 | 3106540 |
| Prevotella enoeca F0113 | 0.066520742 | GCA_001444445.1_ASM144444v1 | 0.032661641 | 0 | 0.967338359 | 2861430 |
| Prevotella intermedia | 1.813605084 | GCA_002763715.1_ASM276371v1 | 0.473325159 | 0 | 0.526674841 | 2764740 |
| KCOM 1949 | | | | | | |
| Prevotella jejuni | 0.065109387 | GCA_002849795.1_ASM284979v1 | 0.026590527 | 0.000144901 | 0.973264571 | 3913010 |
| Prevotella melaninogenica | 0.090290673 | GCA_000144405.1_ASM14440v1 | 0.037824308 | 0.000178961 | 0.96199673 | 3168280 |
| ATCC 25845 ATCC 25845 | | | | | | |
| Prevotella | 0.056076281 | GCA_018127985.1_ASM1812798v1 | 0.027308965 | 0.000187408 | 0.972503628 | 3025490 |
| multiformis F0096 | | | | | | |
| Prevotella nigrescens | 1.416158504 | GCA_018127825.1_ASM1812782v1 | 0.382923408 | 0 | 0.617076592 | 2887110 |
| F0109 | | | | | | |
| Prevotella oris NCTC13071 | 0.078245077 | GCA_900637655.1_52295_B01 | 0.039828799 | 0.000178965 | 0.959992235 | 3168210 |
| Propionibacterium | −0.681330885 | GCA_900087655.1_PFRJS14 | 0 | 0.475049358 | 0.524950642 | 2507190 |
| freudenreichii PFRJS14 | | | | | | |
| Proteus hauseri 15H5D-4a | 0.01861513 | GCA_004116975.1_ASM411697v1 | 0.012499968 | 0.000332763 | 0.987167269 | 3930730 |
| Proteus mirabilis | 0.01794264 | GCA_000069965.1_ASM6996v1 | 0.012599576 | 0.000799776 | 0.986600649 | 4099900 |
| HI4320 HI4320 | | | | | | |
| Proteus terrae subsp. | 0.029997975 | GCA_011045835.1_ASM1104583v1 | 0.017363763 | 0.001002974 | 0.981633263 | 4118750 |
| cibarius ZN2 | | | | | | |
| Providencia alcalifaciens | 0.020651564 | GCA_002393505.1_ASM239350v1 | 0.014393588 | 0.00044886 | 0.985157552 | 3990110 |
| FDAARGOS_408 | | | | | | |
| Providencia heimbachae | 0.016852656 | GCA_900475855.1_46338_B02 | 0.011727718 | 0.000181988 | 0.988090294 | 4286000 |
| NCTC12003 | | | | | | |
| Providencia rettgeri | 0.023141547 | GCA_003204135.1_ASM320413v1 | 0.014860781 | 0.00022698 | 0.984912239 | 4454140 |
| AR_0082 | | | | | | |
| Providencia | 0.023957522 | GCA_010748935.1_ASM1074893v1 | 0.016419628 | 0.001208799 | 0.982371574 | 4432500 |
| vermicola P8538 | | | | | | |
| Rahnella aceris ZF458 | 0.031534324 | GCA_016599695.1_ASM1659969v1 | 0.026052386 | 0.001372841 | 0.972574773 | 5602980 |
| Raoultella electrica | 0.332612782 | GCA_006711645.1_ASM671164v1 | 0.331055798 | 0.006204107 | 0.662740095 | 5785200 |
| DSM 102253 | | | | | | |
| Raoultella ornithinolytica | 0.358851446 | GCA_013457875.1_ASM1345787v1 | 0.370977086 | 0.003511591 | 0.625511322 | 5575250 |
| 172117885 | | | | | | |
| Raoultella planticola | 0.337022176 | GCA_000783935.2_ASM78393v2 | 0.359341373 | 0.0041472 | 0.636511428 | 5823930 |
| FDAARGOS_64 | | | | | | |
| Raoultella terrigena JH01 | 0.429543356 | GCA_012029655.1_ASM1202965v1 | 0.35027463 | 0.004091847 | 0.645633524 | 5598450 |
| Roseburia hominis | −0.089226675 | GCA_902387955.1_UHGG_MGYG- | 0.01867922 | 0.103165819 | 0.878154961 | 3592120 |
| MGYG-HGUT-02517 | | HGUT-02517 | | | | |
| Roseburia intestinalis | −1.313736539 | GCA_900537995.1_Roseburia_intes- | 0.016517075 | 0.571186086 | 0.412296839 | 4493350 |
| L1-82 L1-82 | | tinalis_strain_L1-82 | | | | |
| Ruminococcus albus | −0.019061336 | GCA_000179635.2_ASM17963v2 | 0.00158966 | 0.013551937 | 0.984858403 | 4482090 |
| 7 = DSM 20455 7 | | | | | | |
| Ruminococcus bicirculans 80/3 | −1.078147943 | GCA_000723465.1_Rb803 | 0.00937241 | 0.61342193 | 0.377205659 | 2968500 |
| Salmonella bongori | 0.432500687 | GCA_000439255.1_ASM43925v1 | 0.299470205 | 0.002801694 | 0.697728101 | 4773540 |
| N268-08 N268-08 | | | | | | |
| Scandinavium goeteborgense | 0.293387524 | GCA_003935895.2_ASM393589v2 | 0.207132856 | 0.010936665 | 0.781930479 | 4713960 |
| CCUG 66741 | | | | | | |
| secondary endosymbiont of | 0.016883945 | GCA_000287335.1_ASM28733v1 | 0.010266872 | 0 | 0.989733128 | 1441140 |
| Ctenarytaina | | | | | | |
| eucalypti Ceuc S | | | | | | |
| Serratia ficaria | 0.044111519 | GCA_900187015.1_50465_F01 | 0.036581785 | 0.000875821 | 0.962542395 | 5209970 |
| NCTC12148 | | | | | | |
| Serratia fonticola | 0.038463911 | GCA_001006005.1_ASM100600v1 | 0.03153332 | 0.00105641 | 0.96741027 | 6000510 |
| DSM 4576 | | | | | | |
| Serratia inhibens PRI- | 0.040730131 | GCA_000261045.2_ASM26104v2 | 0.032735552 | 0.001315143 | 0.965949305 | 5474690 |
| 2C PRI-2c | | | | | | |
| Serratia liquefaciens S1 | 0.042947382 | GCA_008364325.2_ASM836432v2 | 0.033771904 | 0.000870289 | 0.965357807 | 5349950 |
| Serratia nematodiphila | 0.049528787 | GCA_004768745.1_ASM476874v1 | 0.038134636 | 0.00077389 | 0.961091474 | 5256560 |
| DH-S01 | | | | | | |
| Serratia plymuthica | 0.039656471 | GCA_000214235.1_ASM21423v1 | 0.032347581 | 0.000995429 | 0.96665699 | 5442880 |
| AS9 AS9 | | | | | | |
| Serratia quinivorans | 0.038931161 | GCA_900638135.1_56433_G01 | 0.032697508 | 0.001617523 | 0.965684969 | 5376740 |
| NCTC13188 | | | | | | |
| Serratia rhizosphaerae | 0.049083103 | GCA_009817885.1_ASM981788v1 | 0.038475299 | 0.00060376 | 0.96092094 | 5098050 |
| KUDC3025 | | | | | | |
| Serratia rubidaea | 0.05037679 | GCA_016026735.1_ASM1602673v1 | 0.040629949 | 0.002813208 | 0.956556844 | 4995010 |
| FDAARGOS_926 | | | | | | |
| Serratia surfactantfaciens | 0.048639398 | GCA_001642805.2_ASM164280v2 | 0.038112294 | 0.000543415 | 0.961344292 | 5117640 |
| YD25 | | | | | | |
| Serratia symbiotica 24.1 | 0.051026543 | GCA_009831665.3_ASM983166v3 | 0.034864509 | 0.000314937 | 0.964820555 | 3210170 |
| Serratia ureilytica T6 | 0.052502986 | GCA_017309605.1_ASM1730960v1 | 0.040394949 | 0.000797188 | 0.958807864 | 5102940 |
| Shigella sonnei SE6-1 | 1.105051116 | GCA_013374815.1_ASM1337481v1 | 0.687118841 | 0.002161767 | 0.310719392 | 4762770 |
| Shimwellia blattae | 0.170234845 | GCA_000262305.1_ASM26230v1 | 0.111187096 | 0.001815703 | 0.886997201 | 4158720 |
| DSM 4481 = NBRC | | | | | | |
| 105725 DSM 4481 | | | | | | |
| Sodaliphilus pleomorphus | 0.02373623 | GCA_009676955.1_ASM967695v1 | 0.011466861 | 0.00197655 | 0.986556589 | 3340670 |
| Oil-RF-744-WCA-WT-10 | | | | | | |
| Sodalis praecaptivus HS1 | 0.026949725 | GCA_000517425.1_ASM51742v1 | 0.018137504 | 0.000529711 | 0.981332785 | 5159420 |
| Solibaculum mannosilyticum | 0.045538573 | GCA_015140235.1_ASM1514023v1 | 0.036432458 | 0.011444164 | 0.952123377 | 2541470 |
| 12CBH8 | | | | | | |
| Streptococcus | −0.09245279 | GCA_001552035.1_ASM155203v1 | 0.004061815 | 0.044190811 | 0.951747374 | 2079120 |
| agalactiae NGBS128 | | | | | | |
| Streptococcus | −0.13021537 | GCA_001412635.1_ASM141263v1 | 0.008721701 | 0.069101745 | 0.922176554 | 1924510 |
| anginosus J4211 | | | | | | |
| Streptococcus canis | −0.066897719 | GCA_010993845.2_ASM1099384v2 | 0.010576932 | 0.04063088 | 0.948792188 | 2157620 |
| HL_77_2 | | | | | | |
| Streptococcus | −0.041359706 | GCA_003086355.2_ASM308635v2 | 0 | 0.025770246 | 0.974229754 | 2443050 |
| chenjunshii Z15 | | | | | | |
| Streptococcus | −0.127756014 | GCA_900475445.1_42727_F01 | 0.002840503 | 0.101844177 | 0.89531532 | 2000350 |
| cristatus ATCC | | | | | | |
| 51100 NCTC12479 | | | | | | |
| Streptococcus | −0.087405664 | GCA_014192895.1_ASM1419289v1 | 0 | 0.043937543 | 0.956062457 | 2111520 |
| dysgalactiae subsp. | | | | | | |
| equisimilis 159 | | | | | | |
| Streptococcus equi | −0.066529025 | GCA_015689455.1_ASM1568945v1 | 0.008321694 | 0.034174814 | 0.957503492 | 2040450 |
| subsp. zooepidemicus SEZ33 | | | | | | |
| Streptococcus ferus | −0.101010101 | GCA_900475025.1_41906_G01 | 0 | 0.047660377 | 0.952339623 | 1872310 |
| NCTC12278 | | | | | | |
| Streptococcus | −0.001414323 | GCA_013267695.1_ASM1326769v1 | 0.000892826 | 0.051034544 | 0.948072631 | 2258000 |
| gallolyticus | | | | | | |
| FDAARGOS_755 | | | | | | |
| Streptococcus | −0.237845797 | GCA_901544385.1_42912_F01 | 0.001059687 | 0.125688271 | 0.873252042 | 2185550 |
| gordonii NCTC10231 | | | | | | |
| Streptococcus | −0.072903523 | GCA_003627155.1_ASM362715v1 | 0.004947579 | 0.106877129 | 0.888175292 | 1972480 |
| gwangjuense ChDC B345 | | | | | | |
| Streptococcus halichoeri | −0.055268078 | GCA_019774635.1_ASM1977463v1 | 0.000649889 | 0.031681717 | 0.967668394 | 2026500 |
| Shali_VAS-CPH | | | | | | |
| Streptococcus | −0.08230517 | GCA_001598035.1_ASM159803v1 | 0 | 0.035968104 | 0.964031896 | 2182100 |
| halotolerans HTS9 | | | | | | |
| Streptococcus | −0.079634351 | GCA_001708305.1_ASM170830v1 | 0 | 0.036784928 | 0.963215072 | 2275470 |
| himalayensis HTS2 | | | | | | |
| Streptococcus | −0.134022181 | GCA_900475975.1_46931_F01 | 0.004420187 | 0.065837192 | 0.929742621 | 1932950 |
| intermedius NCTC11324 | | | | | | |
| Streptococcus | −0.850701276 | GCA_003627135.1_ASM362713v1 | 0.000618033 | 0.337831409 | 0.661550557 | 2009600 |
| koreensis JS71 | | | | | | |
| Streptococcus | −0.706998474 | GCA_018127725.1_ASM1812772v1 | 0.00021265 | 0.275978959 | 0.723808391 | 2144370 |
| lactarius CCUG 66490 | | | | | | |
| Streptococcus | −0.0014788 | GCA_900475675.1_45473_D02 | 0.001030376 | 0.064815001 | 0.934154623 | 1793520 |
| lutetiensis NCTC13774 | | | | | | |
| Streptococcus | −0.075240475 | GCA_001623565.1_ASM162356v1 | 0.000405547 | 0.032993943 | 0.966600511 | 2322790 |
| marmotae HTS5 | | | | | | |
| Streptococcus | −0.050533835 | GCA_900187085.1_50624_E01 | 0 | 0.023392181 | 0.976607819 | 2384130 |
| merionis NCTC13788 | | | | | | |
| Streptococcus mutans | −0.371057021 | GCA_009738105.1_ASM973810v1 | 0 | 0.273052174 | 0.726947826 | 2028030 |
| NCH105 | | | | | | |
| Streptococcus oralis | −0.032312001 | GCA_900637025.1_46338_H01 | 0.014619865 | 0.109078201 | 0.876301934 | 1931550 |
| ATCC 35037 NCTC11427 | | | | | | |
| Streptococcus | −0.043911284 | GCA_001642085.1_ASM164208v1 | 0 | 0.023346272 | 0.976653728 | 2241300 |
| pantholopis TA 26 | | | | | | |
| Streptococcus | −0.049669219 | GCA_018986875.1_ASM1898687v1 | 0.001223865 | 0.027755063 | 0.971021072 | 2193870 |
| parasuis H35 | | | | | | |
| Streptococcus | −0.071441523 | GCA_002900385.1_ASM290038v1 | 0.000694148 | 0.038606036 | 0.960699816 | 2152280 |
| parauberis SPOF3K | | | | | | |
| Streptococcus | −0.001858091 | GCA_004843545.1_ASM484354v1 | 0.003477468 | 0.059026253 | 0.937496279 | 2149840 |
| pasteurianus WUSP067 | | | | | | |
| Streptococcus | −0.135919865 | GCA_003963555.1_ASM396355v1 | 0.008814909 | 0.069695139 | 0.921489952 | 1903820 |
| periodonticum KCOM 2412 | | | | | | |
| Streptococcus | −0.088587389 | GCA_002953735.1_ASM295373v1 | 0.000621635 | 0.043754115 | 0.95562425 | 2065520 |
| pluranimalium TH11417 | | | | | | |
| Streptococcus | −0.058607396 | GCA_901553735.1_41965_D01 | 0 | 0.035626439 | 0.964373561 | 1954700 |
| porcinus NCTC10925 | | | | | | |
| Streptococcus | −0.067451027 | GCA_000221985.1_ASM22198v1 | 0.003823345 | 0.093115794 | 0.903060862 | 2195460 |
| pseudopneumoniae | | | | | | |
| IS7493 IS7493 | | | | | | |
| Streptococcus | −0.034664953 | GCA_900637075.1_48128_D02 | 0.015321002 | 0.034069089 | 0.950609909 | 2156060 |
| pseudoporcinus NCTC13786 | | | | | | |
| Streptococcus | −0.105533374 | GCA_001267845.1_ASM126784v1 | 0 | 0.047913922 | 0.952086078 | 1791400 |
| pyogenes NGAS638 | | | | | | |
| Streptococcus ratti | −0.126156399 | GCA_008803015.1_ASM880301v1 | 0 | 0.086325789 | 0.913674211 | 2096940 |
| ATCC 31377 | | | | | | |
| Streptococcus | −0.073423413 | GCA_003595525.1_ASM359552v1 | 0.00415625 | 0.035105442 | 0.960738309 | 2067970 |
| respiraculi HTS25 | | | | | | |
| Streptococcus | −0.071124561 | GCA_003609975.1_ASM360997v1 | 0 | 0.030791566 | 0.969208434 | 2090540 |
| ruminantium GUT187T | | | | | | |
| Streptococcus | −0.178819072 | GCA_003172975.1_ASM317297v1 | 0 | 0.117138009 | 0.882861991 | 2145290 |
| sobrinus 10919 | | | | | | |
| Streptococcus suis | −0.065836635 | GCA_000026745.1_ASM2674v1 | 0 | 0.034306089 | 0.965693911 | 2170810 |
| BM407 BM407 | | | | | | |
| Streptococcus | −1.093819968 | GCA_903886475.1_Streptoco- | 0 | 0.500912577 | 0.499087423 | 1791630 |
| thermophilus STH_CIRM_65 | | ccus_thermophilus_CIRM_65 | | | | |
| Streptococcus | −0.297896517 | GCA_002355215.1_ASM235521v1 | 0 | 0.220364942 | 0.779635058 | 2097870 |
| troglodytae TKU 31 | | | | | | |
| Streptococcus | −1.064567503 | GCA_900636445.1_41965_G01 | 0 | 0.457611137 | 0.542388863 | 1950300 |
| vestibularis NCTC12167 | | | | | | |
| Tatumella citrea | 0.035594647 | GCA_002163605.1_ASM216360v1 | 0.024061563 | 0.000496996 | 0.97544144 | 4490980 |
| ATCC 39140 | | | | | | |
| Turicibacter sanguinis | −0.467268308 | GCA_013046825.1_ASM1304682v1 | 0 | 0.21909464 | 0.78090536 | 2999690 |
| MOL361 | | | | | | |
| Veillonella dispar | 0.061086815 | GCA_900637515.1_51184_A01 | 0.055002551 | 0.002954764 | 0.942042685 | 2116920 |
| NCTC11831 | | | | | | |
| Veillonella nakazawae T1-7 | 0.071397954 | GCA_013393365.1_ASM1339336v1 | 0.062643601 | 0.005575788 | 0.93178061 | 2097820 |
| Veillonella parvula SKV38 | 0.0975696 | GCA_902810435.1_SKV38 | 0.088362808 | 0 | 0.911637192 | 2146480 |
| Veillonella rodentium | 0.042497383 | GCA_900187285.1_51342_C02 | 0.038557971 | 0.000937642 | 0.960504387 | 2041290 |
| NCTC12018 | | | | | | |
| Xenorhabdus bovienii | 0.021768184 | GCA_000027225.1_ASM2722v1 | 0.013968761 | 0.000239262 | 0.985791977 | 4225500 |
| SS-2004 SS-2004 | | | | | | |
| Xenorhabdus | 0.019982919 | GCA_017743015.1_ASM1774301v1 | 0.013774245 | 0.00023154 | 0.985994215 | 4366410 |
| budapestensis C-7-2 | | | | | | |
| Xenorhabdus | 0.0232173 | GCA_000968195.1_ASM96819v1 | 0.015056201 | 0.00080216 | 0.984141639 | 4203650 |
| doucetiae FRM16 | | | | | | |
| Xenorhabdus | 0.020531365 | GCA_001721185.1_ASM172118v1 | 0.013716143 | 0.000223539 | 0.986060318 | 4522700 |
| hominickii ANU1 | | | | | | |
| Xenorhabdus | 0.020418463 | GCA_000953355.1_XNC2 | 0.013847331 | 0.000347966 | 0.985804703 | 4586660 |
| nematophila AN6/1 | | | | | | |
| Xenorhabdus poinarii G6 G6 | 0.022806567 | GCA_000968175.1_ASM96817v1 | 0.015211011 | 0.000276266 | 0.984512723 | 3659520 |
| Yersinia aldovae 670- | 0.042489601 | GCA_000834395.1_ASM83439v1 | 0.033371057 | 0.000534098 | 0.966094845 | 4471090 |
| 83 670-83 | | | | | | |
| Yersinia canariae | 0.042927907 | GCA_009831415.1_ASM983141v1 | 0.035105251 | 0.001172574 | 0.963722174 | 4710150 |
| NCTC 14382 | | | | | | |
| Yersinia hibernica | 0.041602984 | GCA_004124235.1_ASM412423v1 | 0.034477791 | 0.000521501 | 0.965000708 | 4803440 |
| CFS1934 | | | | | | |
| Yersinia intermedia | 0.041651633 | GCA_009730055.1_ASM973005v1 | 0.033962276 | 0.000570917 | 0.965466807 | 4928910 |
| FDAARGOS 730 | | | | | | |
| Yersinia mollaretii | 0.040370891 | GCA_013282725.1_ASM1328272v1 | 0.031901389 | 0.00073965 | 0.967358961 | 4603530 |
| ATCC 43969 ATCC 43969 | | | | | | |
| Yersinia pestis A1122 | 0.046790684 | GCA_000222975.1_ASM22297v1 | 0.036823723 | 0.000379314 | 0.962796963 | 4658410 |
| A1122 | | | | | | |
| Yersinia pseudotuberculosis | 0.044974385 | GCA_000834295.1_ASM83429v1 | 0.034743348 | 0.00033785 | 0.964918802 | 4839430 |
| IP32953 IP32953 | | | | | | |
| Yersinia rohdei YRA | 0.046264721 | GCA_000834455.1_ASM83445v1 | 0.037077249 | 0.000814455 | 0.962108297 | 4372250 |
| Yersinia ruckeri KMM821 | 0.04275452 | GCA_017498685.1_ASM1749868v1 | 0.034551888 | 0.000405215 | 0.965042897 | 3894230 |
| Yersinia similis 228 | 0.038024271 | GCA_000582515.1_ASM58251v1 | 0.029404098 | 0.000329344 | 0.970266557 | 4964410 |

It will be appreciated that in certain embodiments, any one or any combination of two or more organisms from Table 1 each having a Mean CRC Wald score greater than zero in Column B of Table 1 can be assessed, quantified, or targeted according to the presently disclosed methods, kits, and non-transitory computer readable media.

It will be appreciated that in certain embodiments, any one or any combination of two or more organisms from Table 1 each having a Mean CRC Wald score less than zero in Column B of Table 1 can be assessed, quantified, or promoted.

It will be appreciated that Genome Accession Assembly identifiers as in Column C of Table 1 are available through, for example, the National Institutes of Health (NIH) National Library of Medicine and National Center for Biotechnology Information (see e.g. ncbi.nlm.nih.gov/assembly/, followed by the Genome Accession Assembly identifier)

### Example

In silico discovery was performed by identifying bacterial genes that are consistently observed at higher or lower abundance in CRC patients across four independent global cohorts. Then, cancer-associated and health-associated bacterial consortia were designed, each bacterial consortia containing bacterial isolates that had not previously been linked to CRC. For in vivo validation, gnotobiotic Apc^{Min/+} mice (an established mouse model of CRC) were colonized with the designed bacterial consortia, and tumor burden was quantified.

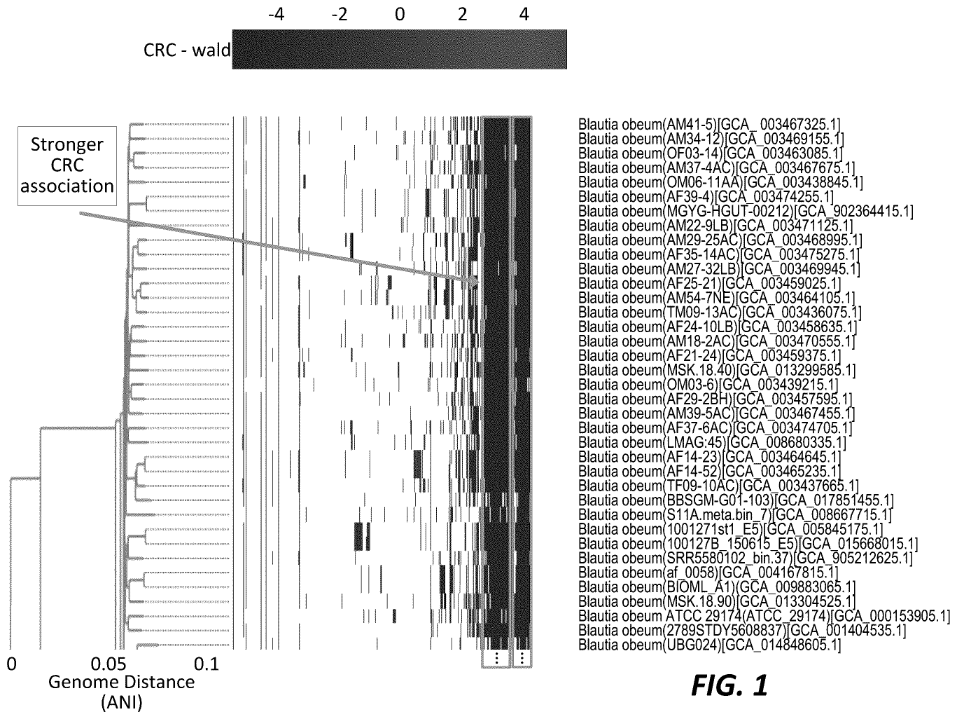

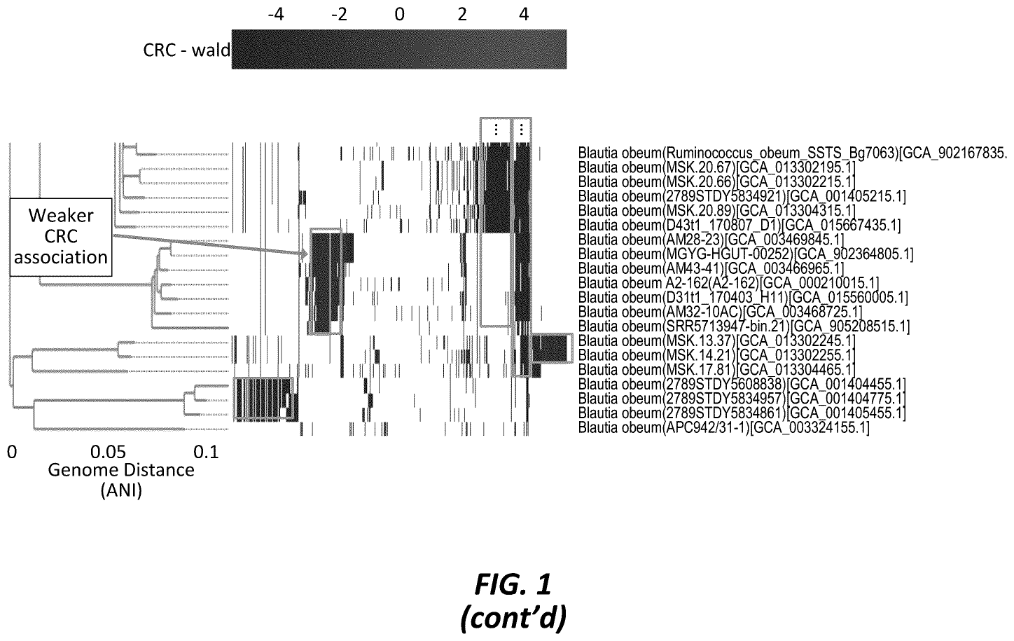

Co-abundant genes (CAGs) can be grouped across a series of metagenomic samples that are identified to be associated with disease, such as CRC (See Nielsen et al, Nature Biotechnology 2014 and Minot et al. Genome Biology 22:135 (2021)). FIG. 1 shows a heat map of identified genes grouped by association with CRC for various strains of Blautia obeum. Gene groupings are outlined. As shown, certain strains of B. obeum share genes that have a stronger CRC association, while certain strains share genes that have a weaker CRC association.

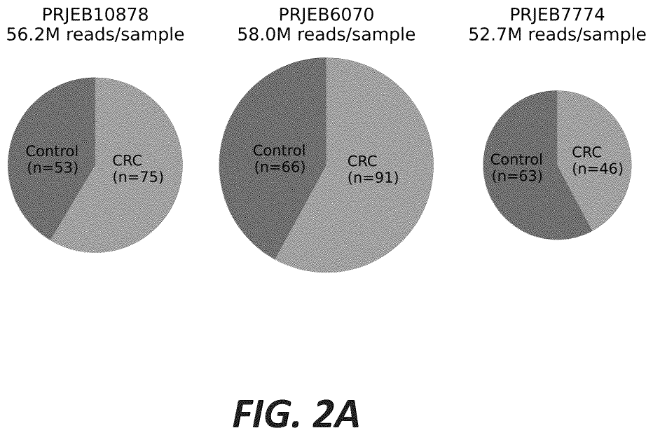

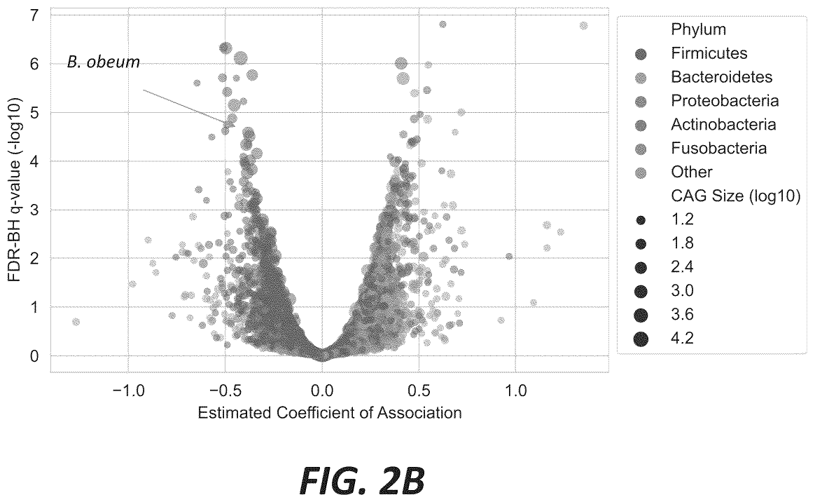

FIGS. 2A and 2B relate to meta-analysis of gut microbiome surveys from global CRC cohorts, pooling published metagenomic datasets. 22,295 CAGs were identified, representing complete and partial microbial genomes reconstructed de novo. Each CAG was tested independently (Martin et al. Ann. Appl. Stat. 14(1): 94-115 (March 2020). DOI: 10.1214/19-AOAS1283) for a significant difference in abundance in CRC across three cohorts (Zeller et al. Molecular Systems Biology 2014; Feng et al. Nature Communications 2015; Yu et al. Gut 2017) and validated in a fourth cohort (Yachida et al. Nature Medicine 2019). 2,319 CAGs were identified, comprising 427,261 genes, that were significantly enriched or depleted in CRC (FDR q<0.2).

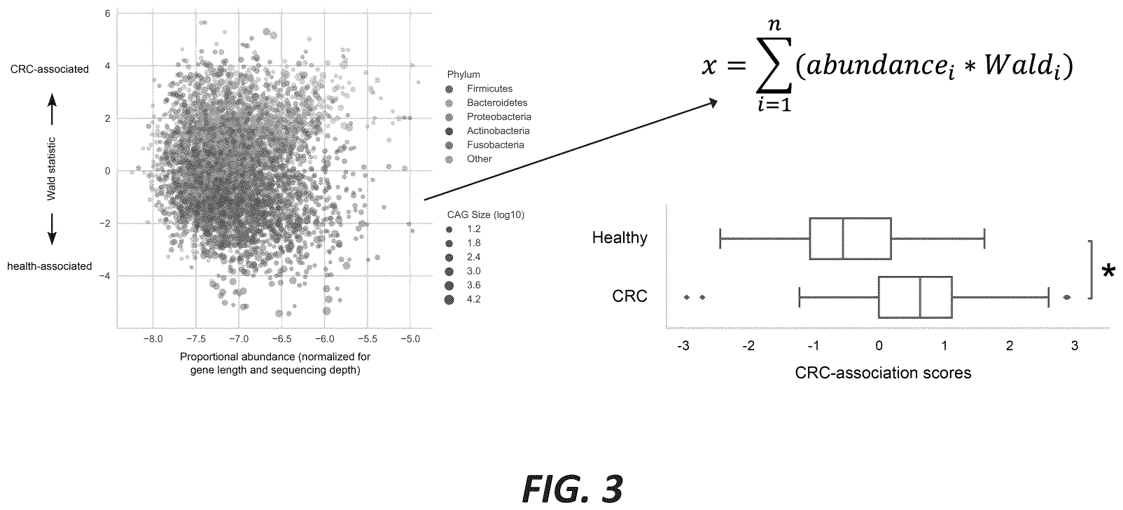

CRC-associated CAGs are encoded in the genomes of phylogenetically diverse bacteria that are observed at varying abundances. FIG. 3 shows Wald statistic association with CRC versus proportional abundance of selected genes (normalized for gene length and sequencing depth), and an example calculation of a CRC-association (Wald) score. Health-associated bacteria represent those exhibiting relatively lower CRC-association scores while CRC-associated bacteria represent this exhibiting relatively higher CRC-association scores. The CRC-association score can be applied to microbiomes or individual bacteria.

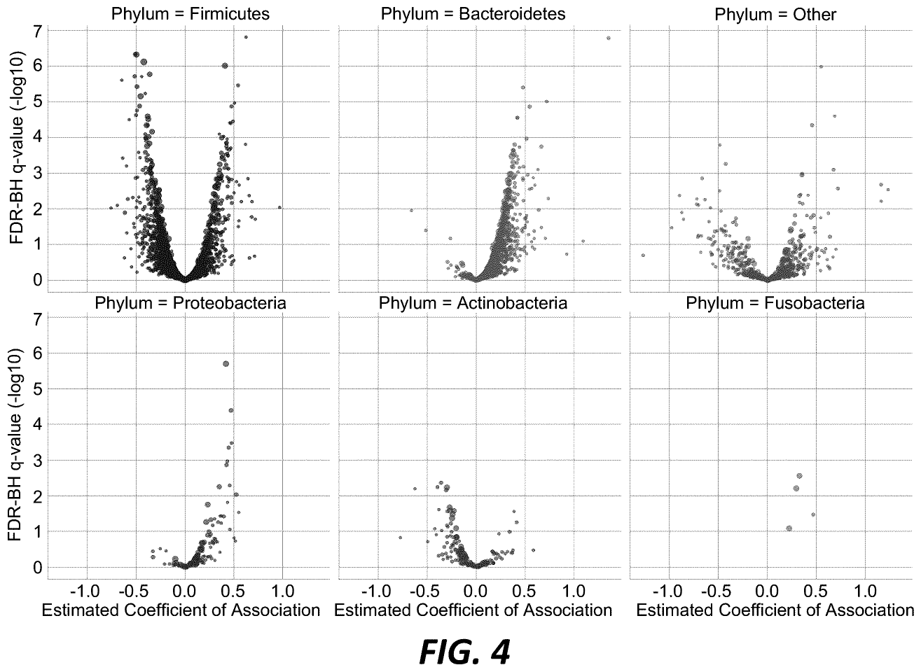

FIG. 4 shows volcano plots revealing taxonomic classification of CAGs. Each CAG was estimated by aligning against the NCBI RefSeq genome collection. Proteobacteria and Bacteroidetes were found at higher abundance in CRC. Firmicutes was found at lower abundance in CRC.

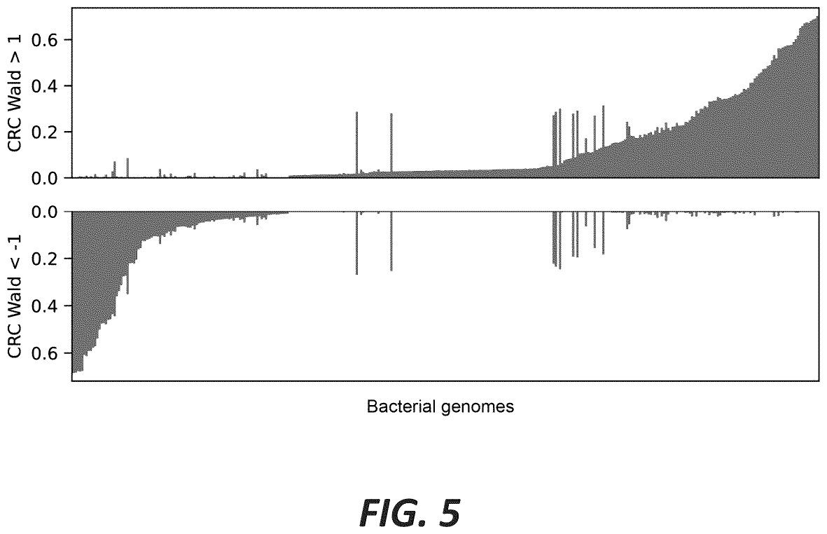

FIG. 5 shows a graph of bacterial genomes of gut bacteria that exhibit a CRC Wald statistic>1 (top) and a CRC Wald statistic<−1 (bottom). The graph indicates that an estimated 10% of gut bacteria harbor genes that are enriched or depleted in CRC.

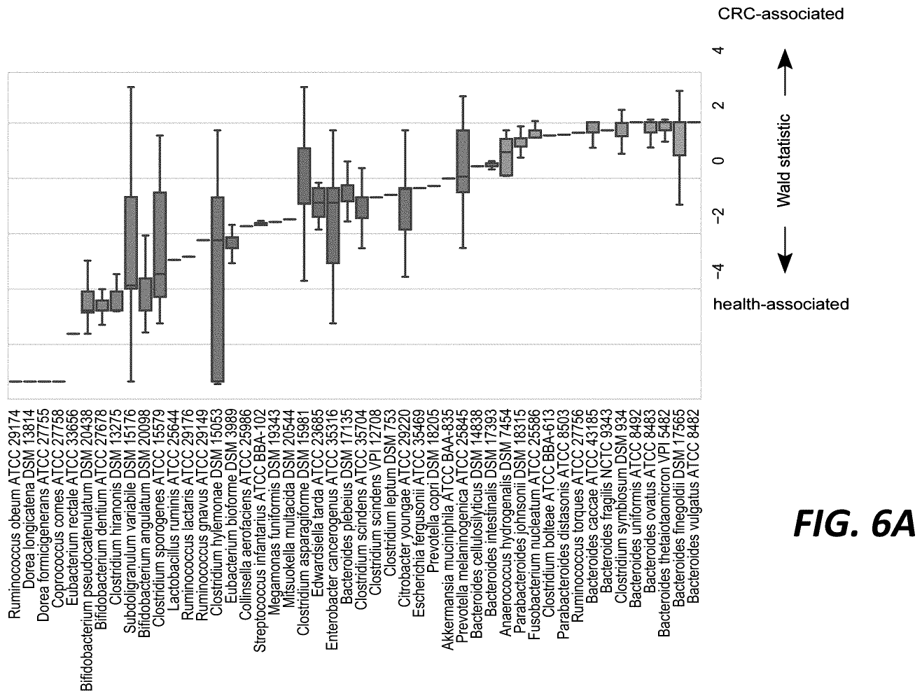

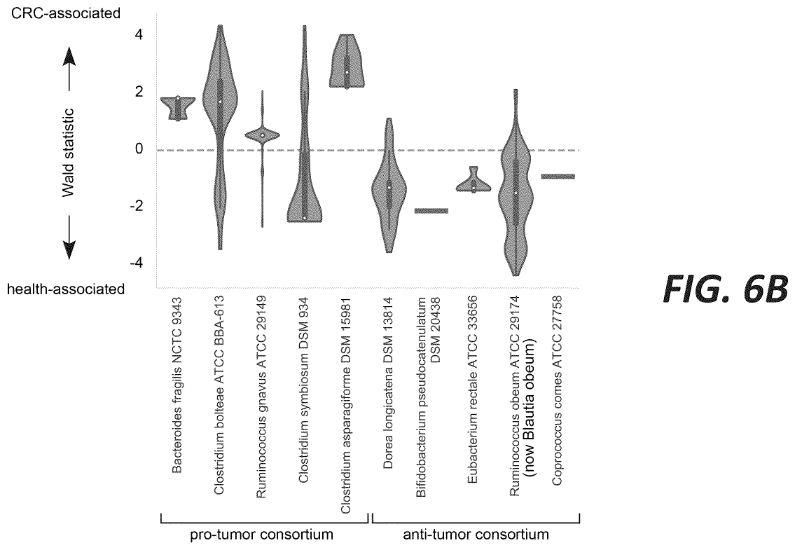

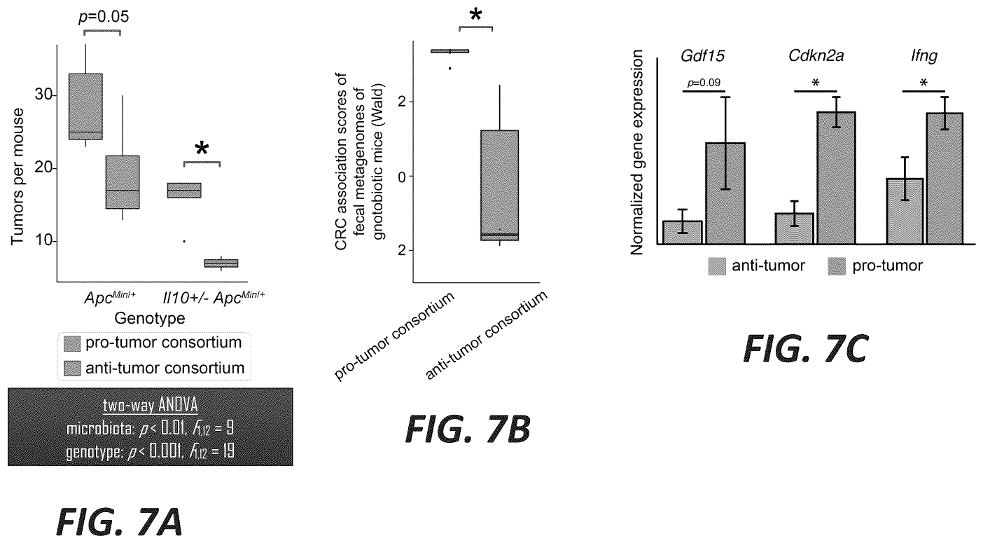

Cancer-associated and health-associated bacterial consortia were designed based on calculated Wald statistics (FIGS. 6A and 6B). Each bacterial consortia contained bacterial isolates that had not previously been linked to CRC. FIGS. 7A-7C show results of introducing the CRC-associated bacterial consortia into a preclinical mouse model of CRC. FIG. 7A shows tumors per mouse versus genotype for a pro-tumor consortium versus an anti-tumor consortium. FIG. 7B shows CRC-association scores (Wald scores) of fecal metagenomes of gnotobiotic mice for the pro-tumor consortium versus the anti-tumor consortium. FIG. 7C shows normalized gene expression for Gdf15, Cdkn2α, and Ifn-g for anti-tumor and pro-tumor consortiums. No direct growth effects on Caco-2 cells were observed in vitro. Gene expression in normal-appearing colonic tissues (lacking any visible tumors) revealed differentially expressed genes involved in senescence.

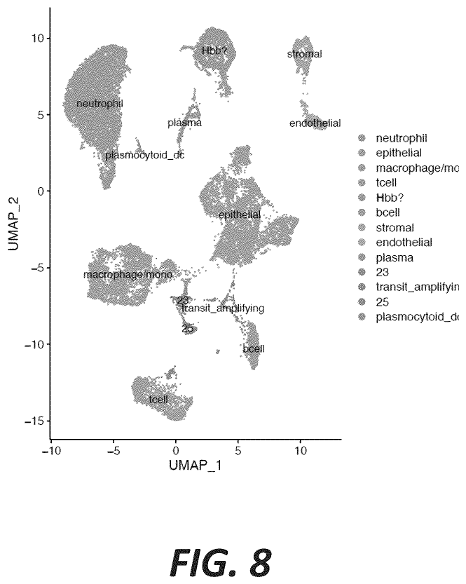

Single-cell RNA sequencing indicated involvement of multiple cell types (FIG. 8). Macrophages and plasma cells were found to express senescence genes. A microbiome-associated alteration in the numbers of B- and T-cells was also observed. Differences in cell numbers combined with unique cell-specific expression patterns resulted in aggregate differences in the senescence tumor signaling pathway.

Table 1 shows a list of 357 genomes from the NCBI Representative Genomes collection with corresponding CRC Association scores. Table 1 includes bacteria species that are health-associated (CRC Wald<0 in Column B) and CRC risk-associated (CRC Wald>0 in Column B). Column A lists organisms and Column B lists Mean Wald scores.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 63/344,523 filed May 20, 2022, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for identifying a subject as being at-risk for developing, as having, or as being at-risk for progressing on colorectal cancer (CRC), the method comprising:
(1) detecting, in a fecal sample from the subject, the presence of one or more organism from Table 1 having a Mean CRC Wald score greater than zero in Column B of Table 1, wherein the subject is identified as at-risk for developing CRC or for progressing on CRC or as having CRC when the one or more organism is present in the fecal sample;
(2) (a) determining whether one or more organism from Table 1 having a Mean CRC Wald score greater than zero in Column B of Table 1 is more abundant in the fecal sample than one or more organism from Table 1 having a Mean CRC Wald score less than zero in Column B of Table 1, and (b) determining that the subject is at-risk for developing or for progressing on CRC or as having CRC when one or more organism from Table 1 having a Mean CRC Wald score greater than zero in Column B of Table 1 is more abundant in the fecal sample than one or more organism from Table 1 having a Mean CRC Wald score less than zero in Column B of Table 1, or
(3) (a) detecting a fecal metagenome in a fecal sample from the subject and (b) comparing (i) the amount or prevalence, in the fecal sample, of one or more organism from Table 1 having a Mean CRC Wald score greater than zero in Column B of Table 1 with (ii) the amount or prevalence of the one or more organism in a reference fecal sample from a non-CRC subject, and/or with (iii) the mean or median amount or prevalence of the one or more organism across a plurality of reference fecal sample from non-CRC subjects, wherein an increase in (i) as compared to (ii) and/or to (iii) identifies the subject as being at-risk for developing for or progressing on CRC or as having CRC.

2.-4. (canceled)

5. A method for treating or managing colorectal cancer (CRC), the method comprising, to a subject identified as being at-risk for developing or for progressing on colorectal cancer (CRC) by the method of claim 1:
(i) prescribing and/or performing a colonoscopy; and/or
(ii) prescribing and/or performing increasing a number and/or a frequency of colonoscopies; and/or
(iii) prescribing and/or performing a colon resection surgery; and/or
(iv) removing one or more polyp; and/or
(v) prescribing a NSAID, such as, for example, aspirin; and/or
(vi) prescribing a plant-based diet or prescribing an increase in the plant content of the subject's diet; and/or
(vii) prescribing and/or administering a compound identified by the method of claim 4; and/or
(viii) manipulating the gut microbiome of the subject, such as, for example, by administering one or more probiotic and/or performing a fecal transplant such that, in a subsequent fecal sample from the subject, the prevalence of one or more organism from Table 1 having a Mean CRC Wald score greater than zero in Column B of Table 1 is decreased relative to the prevalence of one or more organism from Table 1 having a Mean CRC Wald score less than zero in Column B of Table 1, relative to the respective prevalences prior to the manipulation.

6. A method for monitoring colorectal cancer (CRC) in a subject, the method comprising determining whether a fecal sample of the subject comprises (i) a greater or a lesser amount or prevalence of one or more organism from Table 1 having a Mean CRC Wald score greater than zero in Column B of Table 1, as compared to a previous fecal sample from the subject, and/or (ii) an increased or a decreased ratio of [one or more organism from Table 1 having a Mean CRC Wald score greater than zero in Column B of Table 1] to [one or more organism from Table 1 having a Mean CRC Wald score less than zero in Column B of Table 1], as compared to a previous fecal sample from the subject.

7. The method of claim 1, further comprising obtaining the fecal sample from the subject.

8. A kit for identifying a subject as being at-risk for developing, as having, or as being at-risk for progressing on colorectal cancer (CRC), the kit comprising:
(1) a reagent for typing or for identifying one or more organism from Table 1 having a Mean CRC Wald score greater than zero in Column B of Table 1, and, optionally, (2) a reagent for typing or for identifying one or more organism from Table 1 having a mean CRC Wald score less than zero in Column B of Table 1,
wherein the reagent of (1) and/or (2) is optionally selected from the group consisting of:
(i) one or more nucleic acid probe capable of hybridizing with a genomic nucleic acid sequence from one or more organism from Table 1, wherein, preferably, the genomic nucleic acid sequence is present in a Genome Assembly Accession according to Column C of Table 1;
(ii) a forward and a reverse nucleic acid primer capable of amplifying a genomic nucleic acid from one or more organism from Table 1, wherein, preferably, the genomic nucleic acid sequence is present in a Genome Assembly Accession according to Column C of Table 1, and
(iii) one or more antibody specific for the one or more organism from Column B of Table 1; and
instructions for using the reagent(s) to identify the presence or an increased presence of one or more organism from Table 1 having a Mean CRC Wald score greater than zero in Column B of Table 1.

9. The method of claim 1, wherein the one or more organism from Table 1 having a Mean CRC Wald score greater than zero in Column B of Table 1 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more organisms from Table 1 having a Mean CRC Wald score greater than zero in Column B of Table 1.

10. The method of claim 5, wherein the one or more organism from Table 1 having a Mean CRC Wald score less than zero in Column B of Table 1 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more organisms from Table 1 having a Mean CRC Wald score less than zero in Column B of Table 1.

11. The method of claim 1, wherein the one or more organism from Table 1 having a Mean CRC Wald score greater than zero in Column B of Table 1 has a Mean CRC Wald score greater than 0.01, greater than 0.05, greater than 0.1, greater than 0.5, greater than 1, or greater than 2.

12. (canceled)

13. A non-transitory computer readable medium comprising computer executable instructions that when executed cause a processor to:
(1) determine and/or quantify the presence, amount and/or prevalence, in a fecal sample from a subject, of one or more organism from Table 1 (e.g., from Column A of Table 1) having a Mean CRC Wald score greater than zero in Column B of Table 1 (e.g., from Column A of Table 1); and/or
(2) determine and/or quantify the presence, amount and/or prevalence, in a fecal sample from the subject, of one or more organism from Table 1 (e.g., from Column A of Table 1) having a Mean CRC Wald score less than zero in Column B of Table 1 (e.g., from Column A of Table 1), wherein, optionally, the fecal sample of (1) and the fecal sample of (2) are the same sample or were collected from the subject at the same time or were collected from the subject within a 24 hour period.

14. The non-transitory computer readable medium of claim 13, further comprising computer executable instructions that when executed cause a processor (optionally, the processor of claim 13) to generate a ratio of (i) the amount and/or prevalence of the one or more organism from Table 1 having a Mean CRC Wald score greater than zero in Column B of Table 1 to (ii) the amount and/or prevalence of the one or more organism from Table 1 having a Mean CRC Wald score less than zero in Column B of Table 1, in the fecal sample.

15. The non-transitory computer readable medium of claim 13, further comprising computer executable instructions that when executed cause a processor (optionally, the processor of claim 13) to pass an alert to a user that the subject is at-risk for CRC or for progressing on CRC when (a) the presence, amount and/or prevalence, in the fecal sample from a subject, of the one or more organism from Table 1 having a Mean CRC Wald score greater than zero in Column B of Table 1, is greater than: (b) the amount or prevalence of the one or more organism in a reference fecal sample from a non-CRC subject; and/or is greater than (c) the mean or median amount or prevalence of the one or more organism across a plurality of reference fecal sample from non-CRC subjects.

16. The non-transitory computer readable medium of claim 14, further comprising computer executable instructions that when executed cause a processor (optionally, the processor of claim 14) to pass an alert to a user that the subject is at-risk for CRC or for progressing on CRC when the ratio of (i) to (ii) in the fecal sample is greater than: (A) the ratio of (i) to (ii) in a reference fecal sample from a non-CRC subject; and/or (B) (iii) the mean or median ratio of (i) to (ii) across a plurality of reference fecal samples from non-CRC subjects.

17. The non-transitory computer readable medium of claim 14, further comprising computer executable instructions that when executed cause a processor (optionally, the processor of claim 14) to pass an alert to a user that the subject is not at-risk or for CRC or for progressing on CRC when the ratio of (i) to (ii) in the fecal sample is less than: (A) the ratio of (i) to (ii) in a reference fecal sample from a non-CRC subject; and/or (B) (iii) the mean or median ratio of (i) to (ii) across a plurality of reference fecal samples from non-CRC subjects.

18. The non-transitory computer readable medium of claim 15, wherein the user is at least one of a patient and a physician.

19. The non-transitory computer readable medium of claim 15, wherein the alert is provided in at least one of an aural form or a visual form.

20. The non-transitory computer readable medium of claim 15, wherein the alert is indicative of at least one of:
(i) prescribing and/or performing a colonoscopy; and/or
(ii) prescribing and/or performing increasing a number and/or a frequency of colonoscopies; and/or
(iii) prescribing and/or performing a colon resection surgery; and/or
(iv) removing one or more polyp; and/or
(v) prescribing a NSAID, such as aspirin; and/or
(vi) prescribing a plant-based diet or prescribing an increase in the plant content of the subject's diet; and/or
(vii) prescribing and/or administering a compound identified by the method of claim 4; and/or
(viii) manipulating the gut microbiome of the subject, such as, for example, by administering one or more probiotic and/or performing a fecal transplant such that, in a subsequent fecal sample from the subject, the prevalence of one or more organism from Table 1 (e.g., from Column A of Table 1) having a Mean CRC Wald score greater than zero in Column B of Table 1 is decreased relative to the prevalence of one or more organism from Table 1 (e.g., from Column A of Table 1) having a Mean CRC Wald score less than zero in Column B of Table 1, relative to the respective prevalences prior to the manipulation.

21. The non-transitory computer readable medium of claim 13, wherein:
(i) the one or more organism from Table 1 (e.g., from Column A of Table 1) having a Mean CRC Wald score less than zero in Column B of Table 1 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more organisms from Table 1 (e.g., from Column A of Table 1) having a Mean CRC Wald score less than zero in Column B of Table 1; and/or
(ii) the one or more organism from Table 1 (e.g., from Column A of Table 1) having a Mean CRC Wald score greater than zero in Column B of Table 1 comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more organisms from Table 1 (e.g., from Column A of Table 1) having a Mean CRC Wald score greater than zero in Column B of Table 1.

22. The non-transitory computer readable medium of claim 13, further comprising computer executable instructions that when executed cause a processor (optionally, the processor of claim 13) to display a user interface on a display, the user interface having a plurality of fields operable to receive input from a user, the input indicative of whether the subject is at risk of CRC or is at risk of progressing on CRC.
